SEQUENCE LISTING (20 sequences)

SEQ ID NO: 1 - protein, 10 aa, Artificial Sequence
  source: /note=“Description of Artificial Sequence Synthetic peptide”
GYIFSNYWIQ

SEQ ID NO: 2 - protein, 17 aa, Artificial Sequence
  source: /note=“Description of Artificial Sequence Synthetic peptide”
EILPGSGSTEYTENFKD

SEQ ID NO: 3 - protein, 13 aa, Artificial Sequence
  source: /note=“Description of Artificial Sequence Synthetic peptide”
YFFGSSPNWYFDV

SEQ ID NO: 4 - protein, 11 aa, Artificial Sequence
  source: /note=“Description of Artificial Sequence Synthetic peptide”
GASENIYGALN

SEQ ID NO: 5 - protein, 7 aa, Artificial Sequence
  source: /note=“Description of Artificial Sequence Synthetic peptide”
GATNLAD

SEQ ID NO: 6 - protein, 9 aa, Artificial Sequence
  source: /note=“Description of Artificial Sequence Synthetic peptide”
QNVLNTPLT

SEQ ID NO: 7 - protein, 122 aa, Artificial Sequence [sequence omitted]

SEQ ID NO: 8 - protein, 107 aa, Artificial Sequence [sequence omitted]

SEQ ID NO: 9 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 448 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, Artificial Sequence [sequence omitted]

SEQ ID NO: 12 - protein, 122 aa, Artificial Sequence [sequence omitted]

SEQ ID NO: 13 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 14 - protein, 448 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 15 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 448 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 11 aa, Artificial Sequence
  source: /note=“Description of Artificial Sequence Synthetic peptide”
GASENIYHALN

SEQ ID NO: 18 - protein, 17 aa, Artificial Sequence
  source: /note=“Description of Artificial Sequence Synthetic peptide”
EILPGSGHTEYTENFKD

SEQ ID NO: 19 - protein, 10 aa, Artificial Sequence
  source: /note=“Description of Artificial Sequence Synthetic peptide”
GHIFSNYWIQ

SEQ ID NO: 20 - protein, 448 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2023/0002482 A1
(43) Pub. Date: Jan. 5, 2023

(54) HIGH CONCENTRATION ANTI-C5 ANTIBODY FORMULATIONS

(71) Applicant: Alexion Pharmaceuticals, Inc., Boston, MA (US)

(72) Inventors: Leena Philominathan, Cheshire, CT (US); Bruce Mason, Madison, CT (US)

(73) Assignee: Alexion Pharmaceuticals, Inc., Boston, MA (US)

(21) Appl. No.: 17/773,941

(22) PCT Filed: Nov. 4, 2020
(86) PCT No.: PCT/US2020/058779

§ 371 Date: May 3, 2022

Related U.S. Application Data
(60) Provisional application No. 62/932,674, filed on Nov. 8, 2019.
(60) Provisional application No. 62/992,319, filed on Mar. 20, 2020.

(51) Int. Cl. C07K 16/18; A61P 7/02

(52) U.S. Cl. CPC C07K 16/18; A61P 7/02; A61K 2039/505

(57) ABSTRACT

The present disclosure relates to stable aqueous solutions comprising a high concentration of an anti-C5 antibody (e.g., ravulizumab) and methods for preparing the solutions. The disclosure also provides methods for treating or preventing complement-associated disorders, such as paroxysmal nocturnal hemoglobinuria (PNH) and thrombotic microangiopathy (TMA), including atypical hemolytic uremic syndrome (aHUS) using the solutions. Also featured are therapeutic kits containing one or more of the solutions.

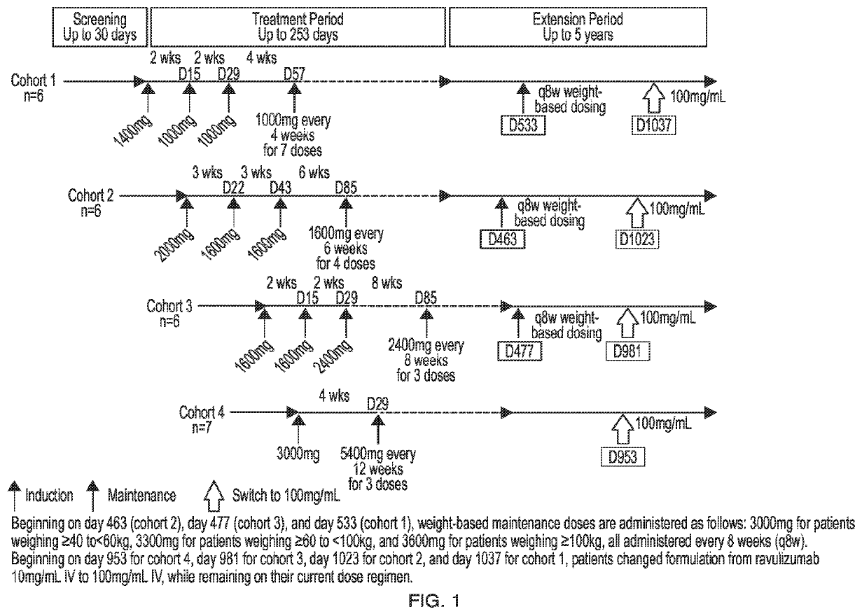

### CROSS REFERENCE TO RELATED APPLICATIONS

This application claims the benefit of U.S. Provisional Application No. 62/932,674, filed Nov. 8, 2019 and U.S. Provisional Application No. 62/992,319, filed Mar. 20, 2020, each of which is incorporated by reference herein in its entirety.

### BACKGROUND

Therapeutic antibodies must be formulated in a way that makes them suitable for administration to patients and maintains their stability during storage and subsequent use. For example, therapeutic antibodies in aqueous solution are prone to degradation, aggregation or undesired chemical modifications, unless the solution is formulated properly. The stability of an antibody in liquid formulation depends on the kinds of excipients used in the formulation, as well as the amounts and proportions of the excipients relative to one another. In addition, viscosity and visual quality must be taken into consideration when preparing an aqueous antibody formulation. Thus, when formulating a therapeutic antibody, considerable care must be taken to devise a formulation that remains stable, contains an adequate concentration of antibody, has a suitable viscosity, and can conveniently be administered to patients.

Anti-C5 antibodies are known in the art. However, there remains a need in the art for novel pharmaceutical formulations comprising anti-C5 antibodies which are sufficiently stable and suitable for administration to patients, e.g., patients with complement-associated disorders, such as PNH or aHUS, who are at risk of substantial morbidity and mortality. Accordingly, it is an object of the present invention to provide improved formulations and methods for treating patients with complement-associated disorders.

### SUMMARY

Provided herein were stable, highly-concentrated, aqueous solutions of an anti-C5 antibody, such as ULTOMIRIS® (ravulizumab) (also known as “antibody BNJ441” and “ALXN1210”), as well as methods for making and using the formulations. The disclosure provides, among other aspects, formulation conditions suitable for maintaining over considerable time the physical and functional stability of ravulizumab in high concentration solutions.

The benefits of stable, highly-concentrated aqueous solutions of an anti-C5 antibody (e.g., ravulizumab), are numerous. First, for therapeutic applications which require the antibody to be administered to a patient in a small volume, therapeutic efficacy often turns on the amount of antibody that can be administered in that small volume.

Furthermore, methods for producing the aqueous solutions described herein do not require a lyophilization step, nor do the featured high concentration aqueous solutions need to be reconstituted from lyophilized material. The instantly featured high concentration antibody solutions provide several advantages over reconstituted lyophilized antibody formulations. First, medical practitioners must locally reconstitute lyophilized antibody solutions aseptically, which increases the opportunity for microbial contamination of the solution prior to administration. In addition, reconstitution requires considerable care to be certain that all the solids contained in the reconstitution vessel were properly dissolved in solution. The high concentration aqueous solutions provided herein thus provide the medical practitioner, caregiver, and/or patient with a fast, easy, safe, and efficient means for delivering a therapeutic antibody to a patient in need thereof.

Other benefits of high concentration formulations include, e.g., manufacturing cost savings from decreasing bulk storage space and/or the number of product fills. In addition, the ability to produce a product having a longer shelf-life will ultimately require fewer production runs, which ultimately reduces cost for the manufacturer and consumer of the highly-concentrated therapeutic antibody.

An exemplary anti-C5 antibody is ravulizumab comprising the heavy and light chains having the sequences shown in SEQ ID NOs: 14 and 11, respectively, or antigen binding fragments and variants thereof. In other embodiments, the antibody comprises the heavy and light chain complementarity determining regions (CDRs) or variable regions (VRs) of ravulizumab. Accordingly, in one embodiment, the antibody comprises the CDR1, CDR2, and CDR3 domains of the heavy chain variable (VH) region of ravulizumab having the sequence shown in SEQ ID NO:12, and the CDR1, CDR2 and CDR3 domains of the light chain variable (VL) region of ravulizumab having the sequence shown in SEQ ID NO:8. In another embodiment, the antibody comprises CDR1, CDR2 and CDR3 heavy chain sequences as set forth in SEQ ID NOs:19, 18, and 3, respectively, and CDR1, CDR2 and CDR3 light chain sequences as set forth in SEQ ID NOs:4, 5, and 6, respectively.

In another embodiment, the antibody comprises VH and VL regions having the amino acid sequences set forth in SEQ ID NO:12 and SEQ ID NO:8, respectively.

In another embodiment, the antibody comprises a heavy chain constant region as set forth in SEQ ID NO:13.

In another embodiment, the antibody comprises a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434 of a native human IgG Fc constant region, each in EU numbering.

In another embodiment, the antibody comprises CDR1, CDR2 and CDR3 heavy chain sequences as set forth in SEQ ID NOs:19, 18, and 3, respectively, and CDR1, CDR2 and CDR3 light chain sequences as set forth in SEQ ID NOs:4, 5, and 6, respectively and a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434 of a native human IgG Fc constant region, each in EU numbering.

In another embodiment, the antibody competes for binding with, and/or binds to the same epitope on C5 as, the above-mentioned antibodies. In another embodiment, the antibody has at least about 90% variable region amino acid sequence identity with the above-mentioned antibodies (e.g., at least about 90%, 91%, 92%, 93%, 94%, 95%, 96%, 97%, 98% or 99% variable region identity with SEQ ID NO:12 and/or SEQ ID NO:8).

In another embodiment, the antibody binds to human C5 at pH 7.4 and 25° C. with an affinity dissociation constant (K_{D}) that is in the range 0.1 nM≤K_{D}≤1 nM. In another embodiment, the antibody binds to human C5 at pH 6.0 and 25° C. with a K_{D}≥10 nM. In yet another embodiment, the [(K_{D} of the antibody or antigen-binding fragment thereof for human C5 at pH 6.0 and at 25° C.)/(K_{D} of the antibody or antigen-binding fragment thereof for human C5 at pH 7.4 and at 25° C.)] of the antibody is greater than 25.

In one aspect, a stable aqueous solution is provided (e.g., a sterile solution). In another embodiment, the stable aqueous solution comprises one or more additional agents (e.g., stabilizing agents, buffering agents, surfactants, and/or preservatives). For example, in one embodiment, the stable aqueous solution comprises a stabilizer. Exemplary stabilizers include, but are not limited to polyols, sugars (e.g., sucrose or trehalose), amino acids (e.g., arginine), amines, and salting out salts. In one embodiment, the solution comprises at least one stabilizing agent at a concentration of 2-10%, inclusive. In another embodiment, the solution comprises at least one or more stabilizing agents at a concentration of 10 mM to 50 mM, inclusive. In another embodiment, the stabilizing agent is present in the solution at a concentration of at least, or equal to, 20 mM. In another embodiment, the stabilizing agent is present in the solution at a concentration of at least, or equal to, 25 mM. In another embodiment, the stabilizing agent is present in the solution at a concentration of at least, or equal to, 50 mM. In another embodiment, the solution comprises 5% sucrose. In another embodiment, the solution comprises between about 30 mg/mL to 60 mg/mL of sucrose (e.g., about 30 mg/mL, 31 mg/mL, 32 mg/mL, 33 mg/mL, 34 mg/ml, 35 mg/ml, 36 mg/mL, 37 mg/mL, 38 mg/mL, 39 mg/mL, 40 mg/mL, 41 mg/mL, 42 mg/mL, 43 mg/mL, 44 mg/mL, 45 mg/mL, 46 mg/mL, 47 mg/mL, 48 mg/mL, 49 mg/mL, 50 mg/mL, 51 mg/mL, 52 mg/mL, 53 mg/mL, 54 mg/mL, 55 mg/mL, 56 mg/mL, 57 mg/mL, 58 mg/mL, 59 mg/mL, or 60 mg/mL). In a particular embodiment, the solution comprises 50 mg/mL of sucrose. In another embodiment, the solution comprises between 20 to 30 mM arginine (e.g., L-arginine). For example, in one embodiment, the solution comprises 20 mM arginine, 21 mM arginine, 22 mM arginine, 23 mM arginine, 24 mM arginine, 25 mM arginine, 26 mM arginine, 27 mM arginine, 28 mM arginine, 29 mM arginine, or 30 mM arginine. In a particular embodiment, the solution comprises 25 mM L-arginine.

In another embodiment, the solution comprises at least one or more buffering agents. Non-limiting examples of typical buffers that can be included in the wash solution(s) include Tris (tris(hydroxymethyl)methylamine), bis-Tris, bis-Tris propane, histidine, triethanolamine, diethanolamine, formate, acetate, MES (2-(N-morpholino)ethanesulfonic acid), phosphate, HEPES (4-2-hydroxyethyl-1-piperazineethanesulfonic acid), citrate, MOPS (3-(N-morpholino)propanesulfonic acid), TAPS (3{[tris(hydroxymethyl)methyl]amino}propanesulfonic acid), Bicine (N,N-bis(2-hydroxyethyl)glycine), Tricine (N-tris(hydroxymethyl)methylglycine), TES (2-{[tris(hydroxymethyl)methyl]amino}ethanesulfonic acid), PIPES (piperazine-N,N′-bis(2-ethanesulfonic acid), cacodylate (dimethylarsinic acid) SSC (saline sodium citrate), sodium phosphate monobasic, and sodium phosphate dibasic. In another embodiment, the buffering agent is an amino acid. The amino acid can be, e.g., one selected from the group consisting of arginine (e.g., L-arginine), histidine (e.g., L-histidine), serine (e.g., L-serine), and glycine (e.g., L-glycine). In another embodiment, the solution comprises two or more buffering agents. In a particular embodiment, the buffering agent is sodium phosphate monobasic. In another particular embodiment, the buffering agent is sodium phosphate dibasic.

In another embodiment, the solution comprises at least one or more buffering agents at a concentration of 10 mM to 300 mM, inclusive. In another embodiment, the solution comprises at least one buffering agent at a concentration of 10 mM to 200 mM, inclusive. In another embodiment, the solution comprises at least one buffering agent at a concentration of 10 mM to 100 mM, inclusive. In another embodiment, the solution comprises at least one buffering agent at a concentration of 10 mM to 50 mM, inclusive. In another embodiment, the solution comprises at least one buffering agent at a concentration of 20 mM to 50 mM, inclusive. In another embodiment, buffering agent is present in the solution at a concentration of at least, or equal to, 20 mM. In another embodiment, buffering agent is present in the solution at a concentration of at least, or equal to, 25 mM. In another embodiment, buffering agent is present in the solution at a concentration of at least, or equal to, 50 mM. In a particular embodiment, the buffering agent is sodium phosphate monobasic at a concentration of about 33.1 mM. In another particular embodiment, the buffering agent is sodium phosphate dibasic at a concentration of about 16.5 mM.

In another embodiment, the solution comprises a surfactant. In one embodiment, the surfactant in the formulation is a non-ionic surfactant. In certain embodiments, the surfactant in the formulation is a polyoxyethylene sorbitan fatty acid ester, for example, polysorbate 20, 40, 60, 80, or a combination of one or more thereof. In one embodiment, the surfactant in the formulation is polysorbate 80 (Tween 80). The concentration of the surfactant in the solution can be, e.g., between 0.001% to 0.02%, inclusive. For example, the surfactant may be present in the formulation in an amount from about 0.001% to about 1%, or about 0.001% to about 0.5%, or about 0.01% to about 0.2%. In one embodiment, the aqueous solutions contain a surfactant at a concentration of at least, or approximately, 0.001 (e.g., at least, or approximately, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 or more) %. In another embodiment, the aqueous solution contains no more than 0.2 (e.g., no more than 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01, 0.009, 0.008, 0.007, 0.006, 0.005, 0.004, 0.003, 0.002, or 0.001) % of a pharmaceutically-acceptable surfactant. In a particular embodiment, the surfactant is 0.05% w/v polysorbate 80.

In another embodiment, the solution comprises water for injection.

In another embodiment, the pH is 7.0, 7.1, 7.2, 7.3, 7.4, 7.5, 7.6, 7.7, 7.8, or 7.9. In another embodiment, the pH of the solution is between 7.0 and 7.4. In another embodiment, the pH of the solution is between 7.2 and 7.8. In another embodiment, the pH of the solution is between 7.2 and 7.6. In a particular embodiment, the pH of the solution is 7.4.

In another embodiment, the solution is in any container suitable for storage of medicines and other therapeutic compositions. For example, the solution may be contained within a sealed and sterilized plastic or glass container having a defined volume, such as a vial, ampule, syringe, cartridge, or bottle. Different types of vials can be used to contain the solutions of the present invention including, e.g., glass or plastic vials. In one embodiment, the vial is a 3 mL single use vial. In another embodiment, the vial is a 3 mL single use vial comprising 300 mg of ravulizumab. In another embodiment, the vial is an 11 mL single use vial. In another embodiment, the vial is an 11 mL single use vial comprising 1,100 mg of ravulizumab.

In another embodiment, the solution is diluted prior to administration. For example, in one embodiment, the solution is diluted prior to administration with normal saline (0.9% sodium chloride). In another embodiment, the solution is diluted prior to administration to a concentration of 50±15 mg/mL ravulizumab (e.g., 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65 mg/mL ravulizumab). In a particular embodiment, the solution is diluted prior to administration to a concentration of about 50 mg/mL ravulizumab. In another embodiment, the solution is stable after dilution for up to 24 hours at 2° C.-8° C. (36° F.-46° F.). In another embodiment, the amount of saline (e.g., sodium chloride) can be optionally decreased, for example, when the solution is administered to a patient on a sodium-controlled (e.g., low sodium) diet.

In one embodiment, the stable aqueous solution comprises no more than seven agents in addition to the anti-C5 antibody, e.g., ravulizumab. In another embodiment, the stable aqueous solution comprises no more than six agents in addition to the anti-C5 antibody, e.g., ravulizumab. In another embodiment, the stable aqueous solution comprises no more than five agents in addition to the anti-C5 antibody, e.g., ravulizumab. In another embodiment, the stable aqueous solution comprises no more than four agents in addition to the anti-C5 antibody, e.g., ravulizumab. In another embodiment, the stable aqueous solution comprises no more than three agents in addition to the anti-C5 antibody, e.g., ravulizumab. In another embodiment, the stable aqueous solution comprises no more than two agents in addition to the anti-C5 antibody, e.g., ravulizumab. In another embodiment, the stable aqueous solution comprises no more than one agent in addition to the anti-C5 antibody, e.g., ravulizumab.

In another embodiment, the stable aqueous solution comprises or consists of ravulizumab at a concentration of 100±20 (e.g., 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120) mg/mL; 50±15 (e.g., 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65) mM phosphate buffer; 5±3 (e.g., 2, 3, 4, 5, 6, 7, or 8) % sucrose; and 25±10 (e.g., 15, 16, 17, 18, 19, 20, 21, 22, 23, 24, 25, 26, 27, 28, 29, 30, 31, 32, 33, 34, or 35) mM arginine; wherein the solution has a pH of 7.4±0.5 (e.g., 6.9, 7, 7.1, 7.2, 7.3, 7.4, 7.5, 7.6, 7.7, 7.8, or 7.9).

In one embodiment, the stable aqueous solution comprises: (a) ravulizumab at a concentration of about 100 mg/mL; (b) about 50 mM sodium phosphate (c) about 0.05% w/v polysorbate 80; (d) about 25 mM L-arginine; (e) about 50 mg/mL sucrose; and (f) water for injection; wherein the solution has a pH of about 7.4 and is contained in a 3 mL single use vial comprising 300 mg of ravulizumab.

In another embodiment, the stable aqueous solution consists of: (a) ravulizumab at a concentration of about 100 mg/mL; (b) about 50 mM sodium phosphate (c) about 0.05% w/v polysorbate 80; (d) about 25 mM L-arginine; (e) about 50 mg/mL sucrose; and (f) water for injection; wherein the solution has a pH of about 7.4 and is contained in a 3 mL single use vial comprising 300 mg of ravulizumab.

In one embodiment, the stable aqueous solution consists of: (a) ravulizumab at a concentration of 100 mg/mL; (b) about 50 mM sodium phosphate (c) 0.05% w/v polysorbate 80; (d) 25 mM L-arginine; (e) 50 mg/mL sucrose; and (f) water for injection; wherein the solution has a pH of about 7.4 and is contained in a 3 mL single use vial comprising 300 mg of ravulizumab.

In one embodiment, the stable aqueous solution comprises: (a) ravulizumab at a concentration of about 100 mg/mL; (b) about 50 mM sodium phosphate (c) about 0.05% w/v polysorbate 80; (d) about 25 mM L-arginine; (e) about 5% w/v sucrose; and (f) water for injection; wherein the solution has a pH of about 7.4 and is contained in a 3 mL single use vial comprising 300 mg of ravulizumab.

In another embodiment, the stable aqueous solution consists of: (a) ravulizumab at a concentration of about 100 mg/mL; (b) about 50 mM sodium phosphate (c) about 0.05% w/v polysorbate 80; (d) about 25 mM L-arginine; (e) about 5% w/v sucrose; and (f) water for injection; wherein the solution has a pH of about 7.4 and is contained in a 3 mL single use vial comprising 300 mg of ravulizumab.

In one embodiment, the stable aqueous solution consists of: (a) ravulizumab at a concentration of 100 mg/mL; (b) about 50 mM sodium phosphate (c) 0.05% w/v polysorbate 80; (d) 25 mM L-arginine; (e) 5% w/v sucrose; and (f) water for injection; wherein the solution has a pH of about 7.4 and is contained in a 3 mL single use vial comprising 300 mg of ravulizumab.

In another embodiment, the stable aqueous solution comprises: (a) ravulizumab at a concentration of about 100 mg/mL; (b) about 33.1 mM sodium phosphate, monobasic; (c) about 16.5 mM sodium phosphate, dibasic; (d) about 0.05% w/v polysorbate 80; (f) about 25 mM L-arginine; (g) about 5% w/v sucrose; and (h) water for injection; wherein the solution has a pH of about 7.4 and is contained in a 3 mL single use vial comprising 300 mg of ravulizumab.

In another embodiment, the stable aqueous solution consists of: (a) ravulizumab at a concentration of about 100 mg/mL; (b) about 33.1 mM sodium phosphate, monobasic; (c) about 16.5 mM sodium phosphate, dibasic; (d) about 0.05% w/v polysorbate 80; (f) about 25 mM L-arginine; (g) about 5% w/v sucrose; and (h) water for injection; wherein the solution has a pH of about 7.4 and is contained in a 3 mL single use vial comprising 300 mg of ravulizumab.

In another embodiment, the stable aqueous solution consists of: (a) ravulizumab at a concentration of 100 mg/mL; (b) 33.1 mM sodium phosphate, monobasic; (c) 16.5 mM sodium phosphate, dibasic; (d) 0.05% w/v polysorbate 80; (f) 25 mM L-arginine; (g) 5% w/v sucrose; and (h) water for injection; wherein the solution has a pH of 7.4 and is contained in a 3 mL single use vial comprising 300 mg of ravulizumab.

In another embodiment, the stable aqueous solution comprises: (a) ravulizumab at a concentration of about 100 mg/mL; (b) about 33.1 mM sodium phosphate, monobasic; (c) about 16.5 mM sodium phosphate, dibasic; (d) about 0.05% w/v polysorbate 80; (f) about 25 mM L-arginine; (g) about 50 mg/mL sucrose; and (h) water for injection; wherein the solution has a pH of about 7.4 and is contained in a 3 mL single use vial comprising 300 mg of ravulizumab.

In another embodiment, the stable aqueous solution consists of: (a) ravulizumab at a concentration of about 100 mg/mL; (b) about 33.1 mM sodium phosphate, monobasic; (c) about 16.5 mM sodium phosphate, dibasic; (d) about 0.05% w/v polysorbate 80; (f) about 25 mM L-arginine; (g) about 50 mg/mL sucrose; and (h) water for injection; wherein the solution has a pH of about 7.4 and is contained in a 3 mL single use vial comprising 300 mg of ravulizumab.

In another embodiment, the stable aqueous solution consists of: (a) ravulizumab at a concentration of 100 mg/mL; (b) 33.1 mM sodium phosphate, monobasic; (c) 16.5 mM sodium phosphate, dibasic; (d) 0.05% w/v polysorbate 80; (f) 25 mM L-arginine; (g) 50 mg/mL sucrose; and (h) water for injection; wherein the solution has a pH of 7.4 and is contained in a 3 mL single use vial comprising 300 mg of ravulizumab.

In another embodiment, the stable aqueous solution comprises: (a) ravulizumab at a concentration of about 100 mg/mL; (b) about 50 mM sodium phosphate (c) about 0.05% w/v polysorbate 80; (d) about 25 mM L-arginine; (e) about 50 mg/mL sucrose; and (f) water for injection, wherein the solution has a pH of about 7.4 and is contained in a 11 mL single use vial comprising 1100 mg of ravulizumab.

In another embodiment, the stable aqueous solution consists of: (a) ravulizumab at a concentration of about 100 mg/mL; (b) about 50 mM sodium phosphate (c) about 0.05% w/v polysorbate 80; (d) about 25 mM L-arginine; (e) about 50 mg/mL sucrose; and (f) water for injection, wherein the solution has a pH of about 7.4 and is contained in a 11 mL single use vial comprising 1100 mg of ravulizumab.

In another embodiment, the stable aqueous solution consists of: (a) ravulizumab at a concentration of 100 mg/mL; (b) about 50 mM sodium phosphate (c) 0.05% w/v polysorbate 80; (d) 25 mM L-arginine; (e) 50 mg/mL sucrose; and (f) water for injection, wherein the solution has a pH of about 7.4 and is contained in a 11 mL single use vial comprising 1100 mg of ravulizumab.

In another embodiment, the stable aqueous solution comprises: (a) ravulizumab at a concentration of about 100 mg/mL; (b) about 50 mM sodium phosphate (c) about 0.05% w/v polysorbate 80; (d) about 25 mM L-arginine; (e) about 5% w/v sucrose; and (f) water for injection, wherein the solution has a pH of about 7.4 and is contained in a 11 mL single use vial comprising 1100 mg of ravulizumab.

In another embodiment, the stable aqueous solution consists of: (a) ravulizumab at a concentration of about 100 mg/mL; (b) about 50 mM sodium phosphate (c) about 0.05% w/v polysorbate 80; (d) about 25 mM L-arginine; (e) about 5% w/v sucrose; and (f) water for injection, wherein the solution has a pH of about 7.4 and is contained in a 11 mL single use vial comprising 1100 mg of ravulizumab.

In another embodiment, the stable aqueous solution consists of: (a) ravulizumab at a concentration of 100 mg/mL; (b) about 50 mM sodium phosphate (c) 0.05% w/v polysorbate 80; (d) 25 mM L-arginine; (e) 5% w/v sucrose; and (f) water for injection, wherein the solution has a pH of about 7.4 and is contained in a 11 mL single use vial comprising 1100 mg of ravulizumab.

In another embodiment, the stable aqueous solution comprises: (a) ravulizumab at a concentration of about 100 mg/mL; (b) about 33.1 mM sodium phosphate, monobasic; (c) about 16.5 mM sodium phosphate, dibasic; (d) about 0.05% w/v polysorbate 80; (f) about 25 mM L-arginine; (g) about 5% w/v sucrose; and (h) water for injection, wherein the solution has a pH of about 7.4 and is contained in a 11 mL single use vial comprising 1100 mg of ravulizumab.

In another embodiment, the stable aqueous solution consists of: (a) ravulizumab at a concentration of about 100 mg/mL; (b) about 33.1 mM sodium phosphate, monobasic; (c) about 16.5 mM sodium phosphate, dibasic; (d) about 0.05% w/v polysorbate 80; (f) about 25 mM L-arginine; (g) about 5% w/v sucrose; and (h) water for injection, wherein the solution has a pH of about 7.4 and is contained in a 11 mL single use vial comprising 1100 mg of ravulizumab.

In another embodiment, the stable aqueous solution consists of: (a) ravulizumab at a concentration of 100 mg/mL; (b) 33.1 mM sodium phosphate, monobasic; (c) 16.5 mM sodium phosphate, dibasic; (d) 0.05% w/v polysorbate 80; (f) 25 mM L-arginine; (g) 5% w/v sucrose; and (h) water for injection; wherein the solution has a pH of 7.4 and is contained in a 11 mL single use vial comprising 1100 mg of ravulizumab.

In another embodiment, the stable aqueous solution comprises: (a) ravulizumab at a concentration of about 100 mg/mL; (b) about 33.1 mM sodium phosphate, monobasic; (c) about 16.5 mM sodium phosphate, dibasic; (d) about 0.05% w/v polysorbate 80; (f) about 25 mM L-arginine; (g) about 50 mg/mL sucrose; and (h) water for injection, wherein the solution has a pH of about 7.4 and is contained in a 11 mL single use vial comprising 1100 mg of ravulizumab.

In another embodiment, the stable aqueous solution consists of: (a) ravulizumab at a concentration of about 100 mg/mL; (b) about 33.1 mM sodium phosphate, monobasic; (c) about 16.5 mM sodium phosphate, dibasic; (d) about 0.05% w/v polysorbate 80; (f) about 25 mM L-arginine; (g) about 50 mg/mL sucrose; and (h) water for injection, wherein the solution has a pH of about 7.4 and is contained in a 11 mL single use vial comprising 1100 mg of ravulizumab.

In another embodiment, the stable aqueous solution consists of: (a) ravulizumab at a concentration of 100 mg/mL; (b) 33.1 mM sodium phosphate, monobasic; (c) 16.5 mM sodium phosphate, dibasic; (d) 0.05% w/v polysorbate 80; (f) 25 mM L-arginine; (g) 50 mg/mL sucrose; and (h) water for injection; wherein the solution has a pH of 7.4 and is contained in a 11 mL single use vial comprising 1100 mg of ravulizumab.

In another embodiment, the stable aqueous solution is sterile. In another embodiment, the solution is a sterile, translucent, clear to yellowish color, preservative-free solution. In another embodiment, the solution has a shelf-life of 18 months.

The solutions described herein can be formulated for any suitable mode of administration. In one embodiment, the solution is formulated for administration by a parenteral mode (e.g., intravenous, subcutaneous, intraperitoneal, or intramuscular injection). In a particular embodiment, the solution is formulated for subcutaneous administration. For example, in one embodiment, the stable aqueous solution comprises an anti-C5 antibody at a concentration of 100 mg/mL and is formulated for subcutaneous administration. In another particular embodiment, the solution is formulated for intravenous administration (e.g., via intravenous infusion). For example, in one embodiment, the stable aqueous solution comprises ravulizumab at a concentration of 100 mg/mL and is formulated for intravenous infusion.

In one embodiment of any of the solutions described herein, the anti-C5 antibody (e.g., ravulizumab) remains at least 95 (e.g., at least 96, 97, 98, or 99)% monomeric during storage at 2° C. to 8° C. for at least six months as determined by SEC-HPLC (e.g., gel permeation HPLC). In another embodiment, the anti-C5 antibody remains at least 95 (e.g., at least 96, 97, 98, or 99)% monomeric during storage at 2° C. to 8° C. for at least nine months as determined by SEC-HPLC. In another embodiment, the anti-C5 antibody remains at least 95 (e.g., at least 96, 97, 98, or 99)% monomeric during storage at 2° C. to 8° C. for at least one year as determined by SEC-HPLC. In another embodiment, the anti-C5 antibody remains at least 95 (e.g., at least 96, 97, 98, or 99)% monomeric during storage at 2° C. to 8° C. for at least 18 months as determined by SEC-HPLC. In another embodiment, the anti-C5 antibody remains at least 95 (e.g., at least 96, 97, 98, or 99)% monomeric during storage at 2° C. to 8° C. for at least two years as determined by SEC-HPLC.

In another embodiment of any of the solutions described herein, less than 5% of the anti-C5 antibody (e.g., ravulizumab) in the solution is aggregated as determined by SEC-HPLC (e.g., gel permeation HPLC). In another embodiment, less than 4% of the anti-C5 antibody in the solution is aggregated as determined by SEC-HPLC. In another embodiment, less than 3% of the anti-C5 antibody in the solution is aggregated as determined by SEC-HPLC. In another embodiment, less than 2% of the anti-C5 antibody in the solution is aggregated as determined by SEC-HPLC. In another embodiment, less than 1% of the anti-C5 antibody in the solution is aggregated as determined by SEC-HPLC.

In another embodiment of any of the solutions described herein, the anti-C5 antibody (e.g., ravulizumab) retains at least 80 (e.g., at least 81, 82, 83, 84, 85, 86, 87, 88, 89, 90, 91, 92, 93, 94, 95, 96, 97, 98, or 99)% of its C5-binding activity during storage at 2° C. to 8° C. for at least six months, as compared to a reference anti-C5 antibody corresponding to the anti-C5 antibody prior to storage. In another embodiment, the anti-C5 antibody (e.g., ravulizumab) retains at least 80 (e.g., at least 81, 82, 83, 84, 85, 86, 87, 88, 89, 90, 91, 92, 93, 94, 95, 96, 97, 98, or 99)% of its C5-binding activity during storage at 2° C. to 8° C. for at least nine months, as compared to a reference anti-C5 antibody corresponding to the anti-C5 antibody prior to storage. In another embodiment, the anti-C5 antibody (e.g., ravulizumab) retains at least 80 (e.g., at least 81, 82, 83, 84, 85, 86, 87, 88, 89, 90, 91, 92, 93, 94, 95, 96, 97, 98, or 99)% of its C5-binding activity during storage at 2° C. to 8° C. for at least one year, as compared to a reference anti-C5 antibody corresponding to the anti-C5 antibody prior to storage. In another embodiment, the anti-C5 antibody (e.g., ravulizumab) retains at least 80 (e.g., at least 81, 82, 83, 84, 85, 86, 87, 88, 89, 90, 91, 92, 93, 94, 95, 96, 97, 98, or 99)% of its C5-binding activity during storage at 2° C. to 8° C. for at least eighteen months, as compared to a reference anti-C5 antibody corresponding to the anti-C5 antibody prior to storage. In another embodiment, the anti-C5 antibody (e.g., ravulizumab) retains at least 80 (e.g., at least 81, 82, 83, 84, 85, 86, 87, 88, 89, 90, 91, 92, 93, 94, 95, 96, 97, 98, or 99)% of its C5-binding activity during storage at 2° C. to 8° C. for at least two years, as compared to a reference anti-C5 antibody corresponding to the anti-C5 antibody prior to storage. In another embodiment, the anti-C5 antibody (e.g., ravulizumab) retains at least 80 (e.g., at least 81, 82, 83, 84, 85, 86, 87, 88, 89, 90, 91, 92, 93, 94, 95, 96, 97, 98, or 99)% of its C5-binding activity during storage at 2° C. to 8° C. for at least three years, as compared to a reference anti-C5 antibody corresponding to the anti-C5 antibody prior to storage.

In another embodiment of any of the solutions described herein, the anti-C5 antibody (e.g., ravulizumab) retains at least 80 (e.g., at least 81, 82, 83, 84, 85, 86, 87, 88, 89, 90, 91, 92, 93, 94, 95, 96, 97, 98, or 99)% of its ability to inhibit hemolysis during storage at 2° C. to 8° C. for at least nine months, as compared to a reference anti-C5 antibody corresponding to the anti-C5 antibody prior to storage. In another embodiment of any of the solutions described herein, the anti-C5 antibody (e.g., ravulizumab) retains at least 80 (e.g., at least 81, 82, 83, 84, 85, 86, 87, 88, 89, 90, 91, 92, 93, 94, 95, 96, 97, 98, or 99)% of its ability to inhibit hemolysis during storage at 2° C. to 8° C. for at least six months, as compared to a reference anti-C5 antibody corresponding to the anti-C5 antibody prior to storage. In another embodiment of any of the solutions described herein, the anti-C5 antibody (e.g., ravulizumab) retains at least 80 (e.g., at least 81, 82, 83, 84, 85, 86, 87, 88, 89, 90, 91, 92, 93, 94, 95, 96, 97, 98, or 99)% of its ability to inhibit hemolysis during storage at 2° C. to 8° C. for at least one year, as compared to a reference anti-C5 antibody corresponding to the anti-C5 antibody prior to storage. In another embodiment of any of the solutions described herein, the anti-C5 antibody (e.g., ravulizumab) retains at least 80 (e.g., at least 81, 82, 83, 84, 85, 86, 87, 88, 89, 90, 91, 92, 93, 94, 95, 96, 97, 98, or 99)% of its ability to inhibit hemolysis during storage at 2° C. to 8° C. for at least 18 months, as compared to a reference anti-C5 antibody corresponding to the anti-C5 antibody prior to storage. In another embodiment of any of the solutions described herein, the anti-C5 antibody (e.g., ravulizumab) retains at least 80 (e.g., at least 81, 82, 83, 84, 85, 86, 87, 88, 89, 90, 91, 92, 93, 94, 95, 96, 97, 98, or 99)% of its ability to inhibit hemolysis during storage at 2° C. to 8° C. for at least two years, as compared to a reference anti-C5 antibody corresponding to the anti-C5 antibody prior to storage. In another embodiment of any of the solutions described herein, the anti-C5 antibody (e.g., ravulizumab) retains at least 80 (e.g., at least 81, 82, 83, 84, 85, 86, 87, 88, 89, 90, 91, 92, 93, 94, 95, 96, 97, 98, or 99)% of its ability to inhibit hemolysis during storage at 2° C. to 8° C. for at least three years, as compared to a reference anti-C5 antibody corresponding to the anti-C5 antibody prior to storage.

Also provided are methods of treating a human patient with a complement-associated condition, comprising administering to the patient a stable aqueous solution (e.g., via intravenous infusion) as described herein, in an amount effective to treat the complement-associated condition. Exemplary complement-associated conditions include, but were not limited to, rheumatoid arthritis, antiphospholipid antibody syndrome, lupus nephritis, ischemia-reperfusion injury, thrombotic microangiopathy (TMA), atypical hemolytic uremic syndrome (aHUS), typical hemolytic uremic syndrome, paroxysmal nocturnal hemoglobinuria (PNH), dense deposit disease, neuromyelitis optica, multifocal motor neuropathy, multiple sclerosis, macular degeneration, HELLP syndrome, spontaneous fetal loss, thrombotic thrombocytopenic purpura, Pauci-immune vasculitis, epidermolysis bullosa, recurrent fetal loss, traumatic brain injury, myocarditis, a cerebrovascular disorder, a peripheral vascular disorder, a renovascular disorder, a mesenteric/enteric vascular disorder, vasculitis, Henoch-Schönlein purpura nephritis, systemic lupus erythematosus-associated vasculitis, vasculitis associated with rheumatoid arthritis, immune complex vasculitis, Takayasu's disease, dilated cardiomyopathy, diabetic angiopathy, Kawasaki's disease, venous gas embolus, restenosis following stent placement, rotational atherectomy, percutaneous transluminal coronary angioplasty, myasthenia gravis, cold agglutinin disease, dermatomyositis, paroxysmal cold hemoglobinuria, antiphospholipid syndrome, Graves' disease, atherosclerosis, Alzheimer's disease, systemic inflammatory response sepsis, septic shock, spinal cord injury, glomerulonephritis, transplant rejection, Hashimoto's thyroiditis, type I diabetes, psoriasis, pemphigus, autoimmune hemolytic anemia, idiopathic thrombocytopenic purpura, Goodpasture's syndrome, Degos disease, and catastrophic antiphospholipid syndrome. In a particular embodiment, the complement-associated condition is aHUS. In another particular embodiment, the complement-associated condition is TMA. In another particular embodiment, the complement-associated condition is PNH.

In one embodiment, a method of treating a human patient is provided, wherein the method comprises administering to the patient a stable aqueous solution based on the weight of the patient. For example, in one embodiment, a method of treating a human patient with PNI-1 is provided, wherein the method comprises administering to the patient a stable aqueous solution (e.g., via intravenous infusion), wherein the stable aqueous solution has a pH of about 7.4 and comprises or consists of ravulizumab at a concentration of about 100 mg/mL, about 33.1 mM sodium phosphate, monobasic, about 16.5 mM sodium phosphate, dibasic, about 0.05% w/v polysorbate 80, about 25 mM L-arginine, about 50 mg/mL sucrose, and water for injection, wherein the stable aqueous solution is administered to the human patient:

(a) on Day 1 at a dose of:
2,400 mg to a patient weighing ≥40 kg to <60 kg,
2,700 mg to a patient weighing ≥60 kg to <100 kg, or
3,000 mg to a patient weighing ≥100 kg;
and

(b) on Day 15 and every eight weeks thereafter at a dose of:
3,000 mg to a patient weighing ≥40 kg to <60 kg,
3,300 mg to a patient weighing ≥60 kg to <100 kg, or
3,600 mg to a patient weighing ≥100 kg.

In one embodiment, the stable aqueous solution is administered to a human patient weighing ≥40 kg to <60 kg on Day 1 at a dose of 2,400 mg and on Day 15 and every eight weeks thereafter at a dose of 3,000 mg. In another embodiment, the aqueous solution is administered to a human patient weighing ≥60 kg to <100 kg on Day 1 at a dose of 2,700 mg and on Day 15 and every eight weeks thereafter at a dose of 3,300 mg. In another embodiment, the stable aqueous solution is administered to a human patient weighing ≥100 kg on Day 1 at a dose of 3,000 mg and on Day 15 and every eight weeks thereafter at a dose of 3,600 mg.

In another embodiment, a method of treating a human patient with aHUS is provided, wherein the method comprises administering to the patient a stable aqueous solution (e.g., via intravenous infusion), wherein the stable aqueous solution has a pH of about 7.4 and comprises or consists of ravulizumab at a concentration of about 100 mg/mL, about 33.1 mM sodium phosphate, monobasic, about 16.5 mM sodium phosphate, dibasic, about 0.05% w/v polysorbate 80, about 25 mM L-arginine, about 50 mg/mL sucrose, and water for injection, wherein the stable aqueous solution is administered to the human patient:

(b) on Day 1 at a dose of:
600 mg to a patient weighing ≥5 kg to <10 kg,
600 mg to a patient weighing ≥10 kg to <20 kg,
900 mg to a patient weighing ≥20 kg to <30 kg;
1,200 mg to a patient weighing ≥30 kg to <40 kg,
2,400 mg to a patient weighing ≥40 kg to <60 kg,
2,700 mg to a patient weighing ≥60 kg to <100 kg, or
3,000 mg to a patient weighing ≥100 kg; and

(b) on Day 15 and every four or eight weeks thereafter at a dose of:
300 mg to a patient weighing ≥5 kg to <10 kg,
600 mg to a patient weighing ≥10 kg to <20 kg,
2,100 mg to a patient weighing ≥20 kg to <30 kg;
2,700 mg to a patient weighing ≥30 kg to <40 kg,
3,000 mg to a patient weighing ≥40 kg to <60 kg,
3,300 mg to a patient weighing ≥60 kg to <100 kg, or
3,600 mg to a patient weighing ≥100 kg.
   In one embodiment, the stable aqueous solution is administered to a human patient weighing ≥5 kg to <10 kg on Day 1 at a dose of 600 mg and on Day 15 and every four weeks thereafter at a dose of 300 mg. In another embodiment, the stable aqueous solution is administered to a human patient weighing ≥10 kg to <20 kg on Day 1 at a dose of 600 mg and on Day 15 and every four weeks thereafter at a dose of 600 mg. In another embodiment, the stable aqueous solution is administered to a human patient weighing ≥20 kg to <30 kg on Day 1 at a dose of 900 mg and on Day 15 and every eight weeks thereafter at a dose of 2,100 mg. In another embodiment, the stable aqueous solution is administered to a human patient weighing ≥30 kg to <40 kg on Day 1 at a dose of 1,200 mg and on Day 15 and every eight weeks thereafter at a dose of 2,700 mg. In another embodiment, the stable aqueous solution is administered to a human patient weighing ≥40 kg to <60 kg on Day 1 at a dose of 2,400 mg and on Day 15 and every eight weeks thereafter at a dose of 3,000 mg. In another embodiment, the stable aqueous solution is administered to a human patient weighing ≥60 kg to <100 kg on Day 1 at a dose of 2,700 mg and on Day 15 and every eight weeks thereafter at a dose of 3,300 mg. In another embodiment, the stable aqueous solution is administered to a human patient weighing ≥100 kg on Day 1 at a dose of 3,000 mg and on Day 15 and every eight weeks thereafter at a dose of 3,600 mg. In another embodiment, the stable aqueous solution is supplied in a 3 mL single use vial comprising 300 mg of ravulizumab. In another embodiment, the stable aqueous solution is supplied in a 11 mL single use vial comprising 1,100 mg of ravulizumab.

In other embodiments, the stable aqueous solution is administered at a flat-fixed dose that is fixed irrespective of the weight of the patient. For example, the stable aqueous solution may be administered at a fixed dose of 1000, 1400, 1600, 1800, 2000, 2400, 3000, or 5400 mg, without regard to the patient's weight. In certain embodiments, dosage regimens are adjusted to provide the optimum desired response (e.g., an effective response).

In another embodiment, the stable aqueous solution is administered to a human patient at a dose of 3000 mg during the induction phase. In another embodiment, the stable aqueous solution is administered to a human patient one time at a dose of 3000 mg during the induction phase. In another embodiment, the stable aqueous solution is administered to a human patient at a dose of 5400 mg during the maintenance phase. In another embodiment, the stable aqueous solution is administered to a human patient three times at a dose of 5400 mg during the maintenance phase. For example, the anti-C5 antibody, or antigen binding fragment thereof, is administered at a dose of 5400 mg on Day 29 of the administration cycle and then every 84 days (or twelve weeks) thereafter during the maintenance phase, i.e., on days 113 and 197 of the administration cycle. In another embodiment, the stable aqueous solution is administered to a human patient at: (a) a dose of 3000 mg on Day 1, (b) a dose of 5400 mg four weeks thereafter, and then (c) at a dose of 5400 mg every 84 days (or twelve weeks) thereafter. In another embodiment, the stable aqueous solution is administered to a human patient at a dose of: (a) 3000 mg on Day 1 of the administration cycle during the induction phase and (b) 5400 mg on Day 29 of the administration cycle and then every 84 days (or twelve weeks) thereafter during the maintenance phase. In another embodiment, the stable aqueous solution is administered to a human patient at a dose of: (a) 3000 mg on Day 1 of the administration cycle during the induction phase and (b) 5400 mg on Days 29, 113, and 197 of the administration cycle during the maintenance phase. In another embodiment, the stable aqueous solution comprises ravulizumab at a concentration of 10 mg/mL. In another embodiment, the stable aqueous solution comprises ravulizumab at a concentration of 100 mg/mL.

The infusion time and rate for administering the 100 mg/mL stable aqueous solution described herein is significantly reduced (e.g., by at least 50%) compared to the infusion time and rate for comparable lower dose (e.g., 10 mg/mL) solutions. The infusion time and rate can be adjusted by the clinician as deemed necessary. In one embodiment, the stable aqueous solution is administered to a human patient weighing ≥5 kg to <10 kg on Day 1 at a dose of 600 mg at a minimum infusion time of 1.4 hours and a maximum infusion rate of 8 mL/hour. In another embodiment, the stable aqueous solution is administered to a human patient weighing ≥5 kg to <10 kg on Day 15 and every four weeks thereafter at a dose of 300 mg at a minimum infusion time of 0.8 hours and a maximum infusion rate of 8 mL/hour. In another embodiment, the stable aqueous solution is administered to a human patient weighing ≥10 kg to <20 kg on Day 1 at a dose of 600 mg at a minimum infusion time of 0.8 hours and a maximum infusion rate of 16 mL/hour. In another embodiment, the stable aqueous solution is administered to a human patient weighing ≥10 kg to <20 kg on Day 15 and every four weeks thereafter at a dose of 600 mg at a minimum infusion time of 0.8 hours and a maximum infusion rate of 16 mL/hour. In another embodiment, the stable aqueous solution is administered to a human patient weighing ≥20 kg to <30 kg on Day 1 at a dose of 900 mg at a minimum infusion time of 0.6 hours and a maximum infusion rate of 30 mL/hour. In another embodiment, the stable aqueous solution is administered to a human patient weighing ≥20 kg to <30 kg on Day 15 and every eight weeks thereafter at a dose of 2,100 mg at a minimum infusion time of 1.3 hours and a maximum infusion rate of 33 mL/hour. In another embodiment, the stable aqueous solution is administered to a human patient weighing ≥30 kg to <40 kg on Day 1 at a dose of 1,200 mg at a minimum infusion time of 0.5 hours and a maximum infusion rate of 46 mL/hour. In another embodiment, the stable aqueous solution is administered to a human patient weighing ≥30 kg to <40 kg on Day 15 and every eight weeks thereafter at a dose of 2,700 mg at a minimum infusion time of 1.1 hours and a maximum infusion rate of 49 mL/hour. In another embodiment, the stable aqueous solution is administered to a human patient weighing ≥40 kg to <60 kg on Day 1 at a dose of 2,400 mg at a minimum infusion time of 0.8 hours and a maximum infusion rate of 64 mL/hour. In another embodiment, the stable aqueous solution is administered to a human patient weighing ≥40 kg to <60 kg on Day 15 and every eight weeks thereafter at a dose of 3,000 mg at a minimum infusion time of 0.9 hours and a maximum infusion rate of 65 mL/hour. In another embodiment, the stable aqueous solution is administered to a human patient weighing ≥60 kg to <100 kg on Day 1 at a dose of 2,700 mg at a minimum infusion time of 0.6 hours and a maximum infusion rate of 92 mL/hour. In another embodiment, the stable aqueous solution is administered to a human patient weighing ≥60 kg to <100 kg on Day 15 and every eight weeks thereafter at a dose of 3,300 mg at a minimum infusion time of 0.7 hours and a maximum infusion rate of 99 mL/hour. In another embodiment, the stable aqueous solution is administered to a human patient weighing ≥100 kg on Day 1 at a dose of 3,000 mg at a minimum infusion time of 0.4 hours and a maximum infusion rate of 144 mL/hour. In another embodiment, the stable aqueous solution is administered to a human patient weighing ≥100 kg on Day 15 and every eight weeks thereafter at a dose of 3,600 mg at a minimum infusion time of 0.5 hours and a maximum infusion rate of 144 mL/hour.

Further provided were kits that include a stable aqueous solution as described herein in a therapeutically effective amount adapted for use in the methods described herein. In one embodiment, the kit comprises: (i) any of the solutions described herein and (ii) instructions for use. In another embodiment, the kit comprises: (i) any of the solutions described herein, (ii) normal saline (0.9% sodium chloride); and (iii) instructions for use. In another embodiment, the kit comprises: (i) a formulation comprising or consisting of ravulizumab at a concentration of about 100 mg/mL; about 33.1 mM sodium phosphate, monobasic; about 16.5 mM sodium phosphate, dibasic; about 0.05% w/v polysorbate 80; about 25 mM L-arginine; and about 50 mg/mL sucrose; (ii) normal saline (0.9% sodium chloride); and (iii) instructions for use. In another embodiment, the formulation is lyophilized. In another embodiment, the kit includes a 3 mL single use vial comprising 300 mg of ravulizumab. In another embodiment, kit includes an 11 mL single use vial comprising 1,100 mg of ravulizumab.

In another aspect, methods for producing a stable concentrated antibody solution are provided. In one embodiment, a method for producing a stable aqueous solution comprising ravulizumab at a concentration of no more than about 10 mg/mL; about 33.1 mM sodium phosphate, monobasic; about 16.5 mM sodium phosphate, dibasic; about 0.05% w/v polysorbate 80; about 25 mM L-arginine; about 50 mg/mL sucrose; and water for injection is provided, wherein the method comprises:

i) providing a first aqueous solution comprising about 10 mg/mL ravulizumab,

ii) subjecting the first aqueous solution to diafiltration into a formulation about 33.1 mM sodium phosphate, monobasic; about 16.5 mM sodium phosphate, dibasic; about 0.05% w/v polysorbate 80; about 25 mM L-arginine; about 50 mg/mL sucrose; and water for injection at pH 7.4 to thereby produce a second aqueous solution; and

iii) concentrating the second aqueous solution to produce a stable aqueous solution comprising ravulizumab at a concentration of about 100 mg/mL; about 33.1 mM sodium phosphate, monobasic; about 16.5 mM sodium phosphate, dibasic; about 0.05% w/v polysorbate 80; about 25 mM L-arginine; about 50 mg/mL sucrose; and water for injection.

### BRIEF DESCRIPTION OF THE DRAWING

The FIGURE is a schematic depicting the design of the Phase II clinical trial (“201 Study”) in PNH patients.

### DETAILED DESCRIPTION

The disclosure features particular stable, aqueous solutions containing a high concentration of anti-C5 antibody (e.g., ravulizumab). The solutions can be used in a variety of therapeutic applications, such as methods for treating or preventing complement-associated disorders. While in no way intended to be limiting, exemplary solutions, formulations, therapeutic kits, and methods for making and using any of the foregoing are elaborated on below and are exemplified in the working Examples.

### I. Definitions

Unless defined otherwise, all technical and scientific terms used herein have the same meaning as commonly understood by the skilled artisan. Although any methods and compositions similar or equivalent to those described herein can be used in practice or testing of the present invention, the preferred methods and compositions are described herein.

The singular form “a,” “an,” and “the” include plural reference unless the context clearly dictates otherwise.

The term “about”, particularly in reference to a given quantity or number, is meant to encompass deviations within plus or minus ten percent (±10%), (e.g., ±5%).

The term “pharmaceutical formulation” refers to preparations which are in such form as to permit the biological activity of the active ingredients to be unequivocally effective, and which contain no additional components which are significantly toxic to the subjects to which the formulation would be administered.

As used herein, an “aqueous” pharmaceutical composition is a composition suitable for pharmaceutical use, wherein the aqueous carrier is water. A composition suitable for pharmaceutical use may be sterile, homogeneous and/or isotonic. Aqueous pharmaceutical compositions may be prepared directly in an aqueous form and/or may be reconstituted from a lyophilisate.

An “isotonic” formulation is one which has essentially the same osmotic pressure as human blood. Isotonic formulations will generally have an osmotic pressure from about 275 to 350 mOsm/kg. The term “hypotonic” describes a formulation with an osmotic pressure below that of human blood. Correspondingly, the term “hypertonic” is used to describe a formulation with an osmotic pressure above that of human blood. Isotonicity can be measured, for example, using a vapor pressure or ice-freezing type osmometer. A “tonicity agent” is a compound which renders the formulation isotonic.

As used herein, the “osmolality” of a solution is the number of osmoles of solute per kilogram of solvent. Osmolality is a measure of the number of particles present in solution and is independent of the size or weight of the particles. It can be measured only by use of a property of the solution that is dependent only on the particle concentration. These properties are vapour pressure depression, freezing point depression, boiling point elevation, and osmotic pressure, and are collectively referred to as colligative properties.

A “sterile” formulation is aseptic or free or essentially free from all living microorganisms and their spores.

A “stable” formulation, as used herein, is one in which the antibody therein essentially retains its physical stability and/or chemical stability and/or biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Stability of the anti-C5 antibody formulations can be measured at selected temperatures after selected time periods. For example, an increase in aggregate formation following storage is an indicator for instability of an aqueous anti-C5 antibody formulation. In addition to aggregate formation, retention of original clarity, color and odor throughout shelf life are indicators utilized to monitor stability of the aqueous anti-C5 antibody solutions described herein.

An antibody “retains its physical stability” in a pharmaceutical formulation if it shows substantially no signs of aggregation, precipitation and/or denaturation upon visual examination of color and/or clarity, or as measured by UV light scattering or by size exclusion chromatography.

The term “aggregation” refers to the assembly of native, folded proteins to from aggregates containing non-native structures. Aggregation can occur even under physiological, non-denaturing conditions, and is often irreversible, resulting in non-native aggregates that are inactive, and sometimes immunogenic and toxic.

The phrase “low to undetectable levels of aggregation” as used herein refers to samples containing no more than about 5%, no more than about 4%, no more than about 3%, no more than about 2%, no more than about 1% and no more than about 0.5% aggregation by weight of protein as measured by gel permeation high-performance liquid chromatography (GP-HPLC), high performance size exclusion chromatography (HPSEC) or static light scattering (SLS) techniques.

An antibody “retains its chemical stability” in a pharmaceutical formulation, if the chemical stability at a given time is such that the antibody is considered to still retain its biological activity as defined below. Chemical stability can be assessed by detecting and quantifying chemically altered forms of the antibody. Chemical alteration may involve size modification (e.g., clipping), deamidation, racemization, hydrolysis, oxidation, beta elimination and disulfide exchange which can be evaluated using known techniques, for example, size exclusion chromatography, SDS-PAGE, matrix-assisted laser desorption ionization/time-of-flight mass spectrometry (MALDI/TOF MS), and/or ion-exchange chromatography.

An antibody “retains its biological activity” in a pharmaceutical formulation, if the antibody in a pharmaceutical formulation is biologically active for its intended purpose. For example, biological activity is retained if the biological activity of the antibody in the pharmaceutical formulation is within about 30%, about 20%, or about 10% (within the errors of the assay) of the biological activity exhibited at the time the pharmaceutical formulation was prepared (e.g., as determined in an antigen binding assay). Herein, “biological activity” of a monoclonal antibody refers to the ability of the antibody to bind to antigen. It can further include antibody binding to antigen and resulting in a measurable biological response which can be measured in vitro or in vivo.

“Shelf-life” of a pharmaceutical product, e.g., an aqueous solution comprising an anti-C5 antibody is the length of time the product is stored before decomposition occurs. For example, shelf-life may be defined as the time for decomposition of 0.1%, 0.5%, 1%, 5%, or 10% of the product.

As used herein, the term “antibody” describes polypeptides comprising at least one antibody derived antigen binding site (e.g., VH/VL region or Fv, or CDR). Antibodies include known forms of antibodies. For example, the antibody can be a human antibody, a humanized antibody, a bispecific antibody, or a chimeric antibody. The antibody also can be a Fab, Fab′2, ScFv, SMIP, Affibody®, nanobody, or a domain antibody. The antibody also can be of any of the following isotypes: IgG1, IgG2, IgG3, IgG4, IgM, IgA1, IgA2, IgAsec, IgD, and IgE. The antibody may be a naturally occurring antibody or may be an antibody that has been altered by a protein engineering technique (e.g., by mutation, deletion, substitution, conjugation to a non-antibody moiety). For example, an antibody may include one or more variant amino acids (compared to a naturally occurring antibody) which changes a property (e.g., a functional property) of the antibody. For example, numerous such alterations are known in the art which affect, e.g., half-life, effector function, and/or immune responses to the antibody in a patient. The term antibody also includes artificial or engineered polypeptide constructs which comprise at least one antibody-derived antigen binding site.

As used herein, the terms “specific binding,” “selective binding,” “selectively binds,” and “specifically binds,” refer to antibody binding to an epitope on a predetermined antigen but not to other antigens. Typically, the antibody (i) binds with an equilibrium dissociation constant (K_{D}) of approximately less than 10⁻⁷ M, such as approximately less than 10⁻⁸ M, 10⁻⁹ M or 10⁻¹⁰ M or even lower when determined by, e.g., surface plasmon resonance (SPR) technology in a BIACORE® 2000 surface plasmon resonance instrument using the predetermined antigen, e.g., C5, as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen. Accordingly, unless otherwise indicated, an antibody that “specifically binds to human C5” refers to an antibody that binds to soluble or cell bound human C5 with a K_{D} of 10⁻⁷ M or less, such as approximately less than 10⁻⁸ M, 10⁻⁹ M or 10⁻¹⁰ M or even lower.

The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jonsson, U., et al. (1993) Ann. Biol. Clin. 51:19-26; Jonsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “K_{off}”, as used herein, is intended to refer to the off rate constant for dissociation of an antibody from the antibody/antigen complex.

The term “K_{d}”, as used herein, is intended to refer to the dissociation constant of a particular antibody-antigen interaction.

As used herein, the terms “subject” or “patient” are used interchangeably herein and refer to a mammal such as a human, mouse, rat, hamster, guinea pig, rabbit, cat, dog, monkey, cow, horse, pig and the like. In one embodiment, the patient is a human patient (e.g., a human patient having a complement-associated condition).

The terms “treat,” “treating,” and “treatment,” as used herein, refer to therapeutic measures described herein. The methods of “treatment” employ administration to a subject the combination disclosed herein in order to cure, delay, reduce the severity of, or ameliorate one or more symptoms of the disease or disorder or recurring disease or disorder, or in order to prolong the survival of a subject beyond that expected in the absence of such treatment.

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method. Effective treatment may refer to alleviation of at least one symptom of a disease or condition.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease or condition, or any other desired alteration of a biological system. In one example, an “effective amount” is the amount of a stable aqueous solution to alleviate at least one symptom of a disease or condition. An effective amount can be administered in one or more administrations.

As used herein, the terms “induction” and “induction phase” are used interchangeably and refer to the first phase of treatment.

As used herein, the terms “maintenance” and “maintenance phase” are used interchangeably and refer to the second phase of treatment. In certain embodiments, treatment is continued as long as clinical benefit is observed or until unmanageable toxicity or disease progression occurs.

### II. Anti-C5 Antibodies

Anti-C5 antibodies (or VH/VL domains derived therefrom) suitable for use in the invention can be generated using methods well known in the art. Alternatively, art recognized anti-C5 antibodies can be used. Antibodies that compete with any of these art-recognized antibodies for binding to C5 also can be used.

An exemplary anti-C5 antibody is ULTOMIRIS® (ravulizumab) comprising heavy and light chains having the sequences shown in SEQ ID NOs:14 and 11, respectively, or antigen binding fragments and variants thereof. Ravulizumab (also known as BNJ441 and ALXN1210) is described in PCT/US2015/019225 and U.S. Pat. No. 9,079,949, the teachings or which are hereby incorporated by reference. The terms ULTOMIRIS®, ravulizumab, BNJ441, and ALXN1210 may be used interchangeably throughout this document. Ravulizumab selectively binds to human complement protein C5, inhibiting its cleavage to C5a and C5b during complement activation. This inhibition prevents the release of the proinflammatory mediator C5a and the formation of the cytolytic pore-forming membrane attack complex (MAC) C5b-9 while preserving the proximal or early components of complement activation (e.g., C3 and C3b) essential for the opsonization of microorganisms and clearance of immune complexes.

In other embodiments, the antibody comprises the heavy and light chain CDRs or variable regions of ravulizumab. Accordingly, in one embodiment, the antibody comprises the CDR1, CDR2, and CDR3 domains of the VH region of ravulizumab having the sequence set forth in SEQ ID NO:12, and the CDR1, CDR2 and CDR3 domains of the VL region of ravulizumab having the sequence set forth in SEQ ID NO:8. In another embodiment, the antibody comprises heavy chain CDR1, CDR2 and CDR3 domains having the sequences set forth in SEQ ID NOs:19, 18, and 3, respectively, and light chain CDR1, CDR2 and CDR3 domains having the sequences set forth in SEQ ID NOs:4, 5, and 6, respectively. In another embodiment, the antibody comprises VH and VL regions having the amino acid sequences set forth in SEQ ID NO:12 and SEQ ID NO:8, respectively.

Another exemplary anti-C5 antibody is antibody BNJ421 comprising heavy and light chains having the sequences shown in SEQ ID NOs:20 and 11, respectively, or antigen binding fragments and variants thereof. BNJ421 (also known as ALXN1211) is described in PCT/US2015/019225 and U.S. Pat. No. 9,079,949, the teachings or which are hereby incorporated by reference.

In other embodiments, the antibody comprises the heavy and light chain CDRs or variable regions of BNJ421. Accordingly, in one embodiment, the antibody comprises the CDR1, CDR2, and CDR3 domains of the VH region of BNJ421 having the sequence set forth in SEQ ID NO:12, and the CDR1, CDR2 and CDR3 domains of the VL region of BNJ421 having the sequence set forth in SEQ ID NO:8. In another embodiment, the antibody comprises heavy chain CDR1, CDR2 and CDR3 domains having the sequences set forth in SEQ ID NOs:19, 18, and 3, respectively, and light chain CDR1, CDR2 and CDR3 domains having the sequences set forth in SEQ ID NOs:4, 5, and 6, respectively. In another embodiment, the antibody comprises VH and VL regions having the amino acid sequences set forth in SEQ ID NO:12 and SEQ ID NO:8, respectively.

The exact boundaries of CDRs have been defined differently according to different methods. In some embodiments, the positions of the CDRs or framework regions within a light or heavy chain variable domain can be as defined by Kabat et al. [(1991) “Sequences of Proteins of Immunological Interest.” NIH Publication No. 91-3242, U.S. Department of Health and Human Services, Bethesda, Md.]. In such cases, the CDRs can be referred to as “Kabat CDRs” (e.g., “Kabat LCDR2” or “Kabat HCDR1”). In some embodiments, the positions of the CDRs of a light or heavy chain variable region can be as defined by Chothia et al. (1989) Nature 342:877-883. Accordingly, these regions can be referred to as “Chothia CDRs” (e.g., “Chothia LCDR2” or “Chothia HCDR3”). In some embodiments, the positions of the CDRs of the light and heavy chain variable regions can be as defined by a Kabat-Chothia combined definition. In such embodiments, these regions can be referred to as “combined Kabat-Chothia CDRs”. Thomas et al. [(1996) Mol Immunol 33(17/18):1389-1401] exemplifies the identification of CDR boundaries according to Kabat and Chothia definitions.

In some embodiments, an anti-C5 antibody described herein comprises a heavy chain CDR1 comprising, or consisting of, the following amino acid sequence: GHIFSNYWIQ (SEQ ID NO:19). In some embodiments, an anti-C5 antibody described herein comprises a heavy chain CDR2 comprising, or consisting of, the following amino acid sequence: EILPGSGHTEYTENFKD (SEQ ID NO:18). In some embodiments, an anti-C5 antibody described herein comprises a heavy chain variable region comprising the following amino acid sequence:

(SEQ ID NO: 12)
QVQLVQSGAEVKKPGASVKVSCKASGHIFSNYWIQWVRQAPGQGLEWMG
EILPGSGHTEYTENFKDRVTMTRDTSTSTVYMELSSLRSEDTAVYYCAR
YFFGSSPNWYFDVWGQGTLVTVSS.

In some embodiments, an anti-C5 antibody described herein comprises a light chain variable region comprising the following amino acid sequence:

(SEQ ID NO: 8)
DIQMTQSPSSLSASVGDRVTITCGASENIYGALNWYQQKPGKAPKLLIY
GATNLADGVPSRFSGSGSGTDFTLTISSLQPEDFATYYCQNVLNTPLTF
GQGTKVEIK.

An anti-C5 antibody described herein can, in some embodiments, comprise a variant human Fc constant region that binds to human neonatal Fc receptor (FcRn) with greater affinity than that of the native human Fc constant region from which the variant human Fc constant region was derived. For example, the Fc constant region can comprise one or more (e.g., two, three, four, five, six, seven, or eight or more) amino acid substitutions relative to the native human Fc constant region from which the variant human Fc constant region was derived. The substitutions can increase the binding affinity of an IgG antibody containing the variant Fc constant region to FcRn at pH 6.0, while maintaining the pH dependence of the interaction. Methods for testing whether one or more substitutions in the Fc constant region of an antibody increase the affinity of the Fc constant region for FcRn at pH 6.0 (while maintaining pH dependence of the interaction) are known in the art and exemplified in the working examples. See, e.g., PCT/US2015/019225 and U.S. Pat. No. 9,079,949 the disclosures of each of which are incorporated herein by reference in their entirety.

Substitutions that enhance the binding affinity of an antibody Fc constant region for FcRn are known in the art and include, e.g., (1) the M252Y/S254T/T256E triple substitution described by Dall'Acqua et al. (2006) J Biol Chem 281: 23514-23524; (2) the M428L or T250Q/M428L substitutions described in Hinton et al. (2004) J Biol Chem 279:6213-6216 and Hinton et al. (2006) J Immunol 176:346-356; and (3) the N434A or T307/E380A/N434A substitutions described in Petkova et al. (2006) Int Immunol 18(12):1759-69. The additional substitution pairings: P257I/Q311I, P257I/N434H, and D376V/N434H are described in, e.g., Datta-Mannan et al. (2007) J Biol Chem 282(3):1709-1717, the disclosure of which is incorporated herein by reference in its entirety.

In some embodiments, the variant constant region has a substitution at EU amino acid residue 255 for valine. In some embodiments, the variant constant region has a substitution at EU amino acid residue 309 for asparagine. In some embodiments, the variant constant region has a substitution at EU amino acid residue 312 for isoleucine. In some embodiments, the variant constant region has a substitution at EU amino acid residue 386.

In some embodiments, the variant Fc constant region comprises no more than 30 (e.g., no more than 29, 28, 27, 26, 25, 24, 23, 22, 21, 20, 19, 18, 17, 16, 15, 14, 13, 12, 11, 10, nine, eight, seven, six, five, four, three, or two) amino acid substitutions, insertions, or deletions relative to the native constant region from which it was derived. In some embodiments, the variant Fc constant region comprises one or more amino acid substitutions selected from the group consisting of: M252Y, S254T, T256E, N434S, M428L, V259I, T250I, and V308F. In some embodiments, the variant human Fc constant region comprises a methionine at position 428 and an asparagine at position 434, each in EU numbering. In some embodiments, the variant Fc constant region comprises a 428L/434S double substitution as described in, e.g., U.S. Pat. No. 8,088,376.

In some embodiments the precise location of these mutations may be shifted from the native human Fc constant region position due to antibody engineering. For example, the 428L/434S double substitution when used in a IgG2/4 chimeric Fc may correspond to 429L and 435S as in the M429L and N435S variants found in ravulizumab (BNJ441) and described in U.S. Pat. No. 9,079,949 the disclosure of which is incorporated herein by reference in its entirety.

In some embodiments, the variant constant region comprises a substitution at amino acid position 237, 238, 239, 248, 250, 252, 254, 255, 256, 257, 258, 265, 270, 286, 289, 297, 298, 303, 305, 307, 308, 309, 311, 312, 314, 315, 317, 325, 332, 334, 360, 376, 380, 382, 384, 385, 386, 387, 389, 424, 428, 433, 434, or 436 (EU numbering) relative to the native human Fc constant region. In some embodiments, the substitution is selected from the group consisting of: methionine for glycine at position 237; alanine for proline at position 238; lysine for serine at position 239; isoleucine for lysine at position 248; alanine, phenylalanine, isoleucine, methionine, glutamine, serine, valine, tryptophan, or tyrosine for threonine at position 250; phenylalanine, tryptophan, or tyrosine for methionine at position 252; threonine for serine at position 254; glutamic acid for arginine at position 255; aspartic acid, glutamic acid, or glutamine for threonine at position 256; alanine, glycine, isoleucine, leucine, methionine, asparagine, serine, threonine, or valine for proline at position 257; histidine for glutamic acid at position 258; alanine for aspartic acid at position 265; phenylalanine for aspartic acid at position 270; alanine, or glutamic acid for asparagine at position 286; histidine for threonine at position 289; alanine for asparagine at position 297; glycine for serine at position 298; alanine for valine at position 303; alanine for valine at position 305; alanine, aspartic acid, phenylalanine, glycine, histidine, isoleucine, lysine, leucine, methionine, asparagine, proline, glutamine, arginine, serine, valine, tryptophan, or tyrosine for threonine at position 307; alanine, phenylalanine, isoleucine, leucine, methionine, proline, glutamine, or threonine for valine at position 308; alanine, aspartic acid, glutamic acid, proline, or arginine for leucine or valine at position 309; alanine, histidine, or isoleucine for glutamine at position 311; alanine or histidine for aspartic acid at position 312; lysine or arginine for leucine at position 314; alanine or histidine for asparagine at position 315; alanine for lysine at position 317; glycine for asparagine at position 325; valine for isoleucine at position 332; leucine for lysine at position 334; histidine for lysine at position 360; alanine for aspartic acid at position 376; alanine for glutamic acid at position 380; alanine for glutamic acid at position 382; alanine for asparagine or serine at position 384; aspartic acid or histidine for glycine at position 385; proline for glutamine at position 386; glutamic acid for proline at position 387; alanine or serine for asparagine at position 389; alanine for serine at position 424; alanine, aspartic acid, phenylalanine, glycine, histidine, isoleucine, lysine, leucine, asparagine, proline, glutamine, serine, threonine, valine, tryptophan, or tyrosine for methionine at position 428; lysine for histidine at position 433; alanine, phenylalanine, histidine, serine, tryptophan, or tyrosine for asparagine at position 434; and histidine for tyrosine or phenylalanine at position 436, all in EU numbering.

Suitable anti-C5 antibodies for use in the methods described herein, in some embodiments, comprise a heavy chain polypeptide comprising the amino acid sequence depicted in SEQ ID NO:14 and/or a light chain polypeptide comprising the amino acid sequence depicted in SEQ ID NO:11. Alternatively, the anti-C5 antibodies for use in the methods described herein, in some embodiments, comprise a heavy chain polypeptide comprising the amino acid sequence depicted in SEQ ID NO:20 and/or a light chain polypeptide comprising the amino acid sequence depicted in SEQ ID NO:11.

In one embodiment, the antibody binds to C5 at pH 7.4 and 25° C. (and, otherwise, under physiologic conditions) with an affinity dissociation constant (K_{D}) that is at least 0.1 (e.g., at least 0.15, 0.175, 0.2, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95, or 0.975) nM. In some embodiments, the K_{D} of the anti-C5 antibody, or antigen binding fragment thereof, is no greater than 1 (e.g., no greater than 0.9, 0.8, 0.7, 0.6, 0.5, 0.4, 0.3, or 0.2) nM.

In other embodiments, the [(K_{D} of the antibody for C5 at pH 6.0 at C)/(K_{D} of the antibody for C5 at pH 7.4 at 25° C.)] is greater than 21 (e.g., greater than 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, 500, 600, 700, 800, 900, 1000, 1500, 2000, 2500, 3000, 3500, 4000, 4500, 5000, 5500, 6000, 6500, 7000, 7500, or 8000).

Methods for determining whether an antibody binds to a protein antigen and/or the affinity for an antibody to a protein antigen are known in the art. For example, the binding of an antibody to a protein antigen can be detected and/or quantified using a variety of techniques such as, but not limited to, Western blot, dot blot, surface plasmon resonance (SPR) method (e.g., BIAcore system; Pharmacia Biosensor AB, Uppsala, Sweden and Piscataway, N.J.), or enzyme-linked immunosorbent assay (ELISA). See, e.g., Benny K. C. Lo (2004) “Antibody Engineering: Methods and Protocols,” Humana Press (ISBN: 1588290921); Johne et al. (1993) J Immunol Meth 160:191-198; Jonsson et al. (1993) Ann Biol Clin 51:19-26; and Jonsson et al. (1991) Biotechniques 11:620-627. In addition, methods for measuring the affinity (e.g., dissociation and association constants) are set forth in the working examples.

As used herein, the term “kₐ” refers to the rate constant for association of an antibody to an antigen. The term “k_{d}” refers to the rate constant for dissociation of an antibody from the antibody/antigen complex. And the term “K_{D}” refers to the equilibrium dissociation constant of an antibody-antigen interaction. The equilibrium dissociation constant is deduced from the ratio of the kinetic rate constants, K_{D}=kₐ/k_{d}. Such determinations preferably are measured at 25° C. or 37° C. (see the working examples). For example, the kinetics of antibody binding to human C5 can be determined at pH 8.0, 7.4, 7.0, 6.5 and 6.0 via surface plasmon resonance (SPR) on a BIAcore 3000 instrument using an anti-Fc capture method to immobilize the antibody.

In one embodiment, the anti-C5 antibody, or antigen binding fragment thereof, blocks the generation or activity of the C5a and/or C5b active fragments of a C5 protein (e.g., a human C5 protein). Through this blocking effect, the antibodies inhibit, e.g., the pro-inflammatory effects of C5a and the generation of the C5b-9 membrane attack complex (MAC) at the surface of a cell.

Methods for determining whether a particular antibody described herein inhibits C5 cleavage are known in the art. Inhibition of human complement component C5 can reduce the cell-lysing ability of complement in a subject's body fluids. Such reductions of the cell-lysing ability of complement present in the body fluid(s) can be measured by methods well known in the art such as, for example, by a conventional hemolytic assay such as the hemolysis assay described by Kabat and Mayer (eds.), “Experimental Immunochemistry, 2^{nd} Edition,” 135-240, Springfield, Ill., CC Thomas (1961), pages 135-139, or a conventional variation of that assay such as the chicken erythrocyte hemolysis method as described in, e.g., Hillmen et al. (2004) N Engl J Med 350(6):552. Methods for determining whether a candidate compound inhibits the cleavage of human C5 into forms C5a and C5b are known in the art and described in Evans et al. (1995) Mol Immunol 32(16):1183-95. For example, the concentration and/or physiologic activity of C5a and C5b in a body fluid can be measured by methods well known in the art. For C5b, hemolytic assays or assays for soluble C5b-9 as discussed herein can be used. Other assays known in the art can also be used. Using assays of these or other suitable types, candidate agents capable of inhibiting human complement component C5 can be screened.

Immunological techniques such as, but not limited to, ELISA can be used to measure the protein concentration of C5 and/or its split products to determine the ability of an anti-C5 antibody, or antigen binding fragment thereof, to inhibit conversion of C5 into biologically active products. In some embodiments, C5a generation is measured. In some embodiments, C5b-9 neoepitope-specific antibodies are used to detect the formation of terminal complement.

Hemolytic assays can be used to determine the inhibitory activity of an anti-C5 antibody, or antigen binding fragment thereof, on complement activation. In order to determine the effect of an anti-C5 antibody, or antigen binding fragment thereof, on classical complement pathway-mediated hemolysis in a serum test solution in vitro, for example, sheep erythrocytes coated with hemolysin or chicken erythrocytes sensitized with anti-chicken erythrocyte antibody are used as target cells. The percentage of lysis is normalized by considering 100% lysis equal to the lysis occurring in the absence of the inhibitor. In some embodiments, the classical complement pathway is activated by a human IgM antibody, for example, as utilized in the Wieslab® Classical Pathway Complement Kit (Wieslab® COMPL CP310, Euro-Diagnostica, Sweden). Briefly, the test serum is incubated with an anti-C5 antibody, or antigen binding fragment thereof, in the presence of a human IgM antibody. The amount of C5b-9 that is generated is measured by contacting the mixture with an enzyme conjugated anti-C5b-9 antibody and a fluorogenic substrate and measuring the absorbance at the appropriate wavelength. As a control, the test serum is incubated in the absence of the anti-C5 antibody, or antigen binding fragment thereof. In some embodiments, the test serum is a C5-deficient serum reconstituted with a C5 polypeptide.

To determine the effect of an anti-C5 antibody, or antigen binding fragment thereof, on alternative pathway-mediated hemolysis, unsensitized rabbit or guinea pig erythrocytes can be used as the target cells. In some embodiments, the serum test solution is a C5-deficient serum reconstituted with a C5 polypeptide. The percentage of lysis is normalized by considering 100% lysis equal to the lysis occurring in the absence of the inhibitor. In some embodiments, the alternative complement pathway is activated by lipopolysaccharide molecules, for example, as utilized in the Wieslab® Alternative Pathway Complement Kit (Wieslab® COMPL AP330, Euro-Diagnostica, Sweden). Briefly, the test serum is incubated with an anti-C5 antibody, or antigen binding fragment thereof, in the presence of lipopolysaccharide. The amount of C5b-9 that is generated is measured by contacting the mixture with an enzyme conjugated anti-C5b-9 antibody and a fluorogenic substrate and measuring the fluorescence at the appropriate wavelength. As a control, the test serum is incubated in the absence of the anti-C5 antibody, or antigen binding fragment thereof.

In some embodiments, C5 activity, or inhibition thereof, is quantified using a CH50 eq assay. The CH50 eq assay is a method for measuring the total classical complement activity in serum. This test is a lytic assay, which uses antibody-sensitized erythrocytes as the activator of the classical complement pathway and various dilutions of the test serum to determine the amount required to give 50% lysis (CH50). The percent hemolysis can be determined, for example, using a spectrophotometer. The CH50 eq assay provides an indirect measure of terminal complement complex (TCC) formation, since the TCC themselves are directly responsible for the hemolysis that is measured.

The assay is well known and commonly practiced by those of skill in the art. Briefly, to activate the classical complement pathway, undiluted serum samples (e.g., reconstituted human serum samples) are added to microassay wells containing the antibody-sensitized erythrocytes to thereby generate TCC. Next, the activated sera are diluted in microassay wells, which are coated with a capture reagent (e.g., an antibody that binds to one or more components of the TCC). The TCC present in the activated samples bind to the monoclonal antibodies coating the surface of the microassay wells. The wells are washed and to each well is added a detection reagent that is detectably labeled and recognizes the bound TCC. The detectable label can be, e.g., a fluorescent label or an enzymatic label. The assay results are expressed in CH50 unit equivalents per milliliter (CH50 U Eq/mL).

Inhibition, e.g., as it pertains to terminal complement activity, includes at least a 5 (e.g., at least a 6, 7, 8, 9, 10, 15, 20, 25, 30, 35, 40, 45, 50, 55, or 60) % decrease in the activity of terminal complement in, e.g., a hemolytic assay or CH50 eq assay as compared to the effect of a control antibody (or antigen-binding fragment thereof) under similar conditions and at an equimolar concentration. Substantial inhibition, as used herein, refers to inhibition of a given activity (e.g., terminal complement activity) of at least 40 (e.g., at least 45, 50, 55, 60, 65, 70, 75, 80, 85, 90, or 95 or greater) %. In some embodiments, an anti-C5 antibody described herein contains one or more amino acid substitutions relative to the CDRs of eculizumab (i.e., SEQ ID NOs:1-6), yet retains at least 30 (e.g., at least 31, 32, 33, 34, 35, 36, 37, 38, 39, 40, 41, 42, 43, 44, 45, 46, 47, 48, 49, 50, 55, 60, 65, 70, 75, 80, 85, 90, or 95) % of the complement inhibitory activity of eculizumab in a hemolytic assay or CH50 eq assay.

An anti-C5 antibody described herein has a serum half-life in humans that is at least 20 (e.g., at least 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55) days. In another embodiment, the anti-C5 antibody described herein has a serum half-life in humans that is at least 40 days. In another embodiment, the anti-C5 antibody described herein has a serum half-life in humans that is approximately 43 days. In another embodiment, the anti-C5 antibody described herein has a serum half-life in humans that is between 39-48 days. Methods for measuring the serum half-life of an antibody are known in the art. In some embodiments, an anti-C5 antibody, or antigen binding fragment thereof, described herein has a serum half-life that is at least 20 (e.g., at least 30, 35, 40, 45, 50, 55, 60, 65, 70, 75, 80, 85, 90, 95, 100, 125, 150, 175, 200, 250, 300, 400, 500) % greater than the serum half-life of eculizumab, e.g., as measured in one of the mouse model systems described in the working examples (e.g., the C5-deficient/NOD/scid mouse or hFcRn transgenic mouse model system).

In one embodiment, the antibody competes for binding with, and/or binds to the same epitope on C5 as, the antibodies described herein. The term “binds to the same epitope” with reference to two or more antibodies means that the antibodies bind to the same segment of amino acid residues, as determined by a given method. Techniques for determining whether antibodies bind to the “same epitope on C5” with the antibodies described herein include, for example, epitope mapping methods, such as, x-ray analyses of crystals of antigen:antibody complexes which provides atomic resolution of the epitope and hydrogen/deuterium exchange mass spectrometry (HDX-MS). Other methods monitor the binding of the antibody to peptide antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same VH and VL or the same CDR1, 2 and 3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In certain embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the cold antibody that is incubated first with the target). Competing antibodies bind to the same epitope, an overlapping epitope or to adjacent epitopes (e.g., as evidenced by steric hindrance).

Anti-C5 antibodies, or antigen-binding fragments thereof described herein, used in the methods described herein can be generated using a variety of art-recognized techniques. Monoclonal antibodies may be obtained by various techniques familiar to those skilled in the art. Briefly, spleen cells from an animal immunized with a desired antigen are immortalized, commonly by fusion with a myeloma cell (see, Kohler & Milstein, Eur. J. Immunol. 6: 511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al., Science 246: 1275-1281 (1989).

### III. Highly-Concentrated Anti-C5 Antibody Solutions

Provided herein are stable aqueous solutions comprising an anti-C5 antibody (e.g., ravulizumab). The aqueous solutions described herein can be sterile, pharmaceutical-grade compositions, e.g., for administration to a subject for the treatment or prevention of a complement-associated disorder, such as PNH, TMA, or aHUS. The solutions described herein can be formulated according to standard methods. Pharmaceutical formulation is a well-established art, and is further described in, e.g., Gennaro (2000) “Remington: The Science and Practice of Pharmacy,” 20^{th} Edition, Lippincott, Williams & Wilkins (ISBN: 0683306472); Ansel et al. (1999) “Pharmaceutical Dosage Forms and Drug Delivery Systems,” 7^{th} Edition, Lippincott Williams & Wilkins Publishers (ISBN: 0683305727); and Kibbe (2000) “Handbook of Pharmaceutical Excipients American Pharmaceutical Association,” 3^{rd} Edition (ISBN: 091733096X). Suitable formulation methods for the high concentration antibody solutions described herein are exemplified in the working examples.

The aqueous solutions described herein comprise a high concentration of an antibody that binds to human complement component C5, such as ravulizumab. Such solutions are sometimes referred to herein as “high concentration antibody solutions.” As used herein, a “high concentration” of an anti-C5 antibody (e.g., ravulizumab) in an aqueous solution is a concentration of the antibody that is at least, equal to, or greater than, 40 (e.g., at least, equal to, or greater than, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300) mg/mL. In one embodiment, the anti-C5 antibody is present in the solution at a concentration of more than 100 (e.g., more than 105, 110, 115, 120, 125, 130, 135, 140, 145, 150, 155, 160, 165, 170, 175, 180, 185, 190, or 195) mg/mL. In another embodiment, the anti-C5 antibody is present in the solution at a concentration of more than 200 (e.g., more than 200, 205, 210, 215, 220, 225, 230, 235, 240, 245, 250, 255, 260, 265, 270, 275, 280, 285, 290, or 295) mg/mL. In another embodiment, the anti-C5 antibody is present in the solution at a concentration of more than 300 mg/mL. In another embodiment, the antibody is present in the solution at a concentration of, e.g., 40 mg/mL to 200 mg/mL, 50 mg/mL to 200 mg/mL, 60 mg/mL to 200 mg/mL, 70 mg/mL to 200 mg/mL, 80 mg/mL to 200 mg/mL, 90 mg/mL to 200 mg/mL, 100 mg/mL to 200 mg/mL, 110 mg/mL to 200 mg/mL, 120 mg/mL to 200 mg/mL, 130 mg/mL to 200 mg/mL, 140 mg/mL to 200 mg/mL, 150 mg/mL to 200 mg/mL, 40 mg/mL to 100 mg/mL, 50 mg/mL to 100 mg/mL, 60 mg/mL to 100 mg/mL, 70 mg/mL to 100 mg/mL, 80 mg/mL to 100 mg/mL, 90 mg/mL to 100 mg/mL, 40 mg/mL to 150 mg/mL, 50 mg/mL to 150 mg/mL, 60 mg/mL to 150 mg/mL, 70 mg/mL to 150 mg/mL, 80 mg/mL to 150 mg/mL, 90 mg/mL to 150 mg/mL, 100 mg/mL to 150 mg/mL, 110 mg/mL to 150 mg/mL, 120 mg/mL to 150 mg/mL, 40 mg/mL to 50 mg/mL, 40 mg/mL to 250 mg/mL, 50 mg/mL to 250 mg/mL, 60 mg/mL to 250 mg/mL, 70 mg/mL to 250 mg/mL, 80 mg/mL to 250 mg/mL, 90 mg/mL to 250 mg/mL, 100 mg/mL to 250 mg/mL, 110 mg/mL to 250 mg/mL, 120 mg/mL to 250 mg/mL, 130 mg/mL to 250 mg/mL, 140 mg/mL to 250 mg/mL, 150 mg/mL to 250 mg/mL, 160 mg/mL to 250 mg/mL, 170 mg/mL to 250 mg/mL, 180 mg/mL to 250 mg/mL, 190 mg/mL to 250 mg/mL, 200 mg/mL to 250 mg/mL, greater than 200 mg/mL (e.g., at least 201 mg/mL) to 250 mg/mL, or greater than 200 mg/mL (e.g., 201 mg/mL or greater) to 300 mg/mL.

The featured aqueous solutions provide the anti-C5 antibody (e.g., ravulizumab) formulated therein with marked physical and chemical stability, as well as functional stability. For example, the formulations described herein are capable of maintaining the structural integrity of an anti-C5 antibody (e.g., ravulizumab) present at high concentrations in a solution. In one embodiment, the solution is suitable for storage at 2-8° C. (e.g., 4° C.). In another embodiment, the solution is formulated for storage at a temperature below 0° C. (e.g., −20° C. or −80° C.). In another embodiment, the solution is formulated for storage for up to three years (e.g., one month, two months, three months, four months, five months, six months, seven months, eight months, nine months, 10 months, 11 months, 1 year, 1½ years, 2 years, 2½ years, or 3 years) at 2-8° C. (e.g., 4° C.). In another embodiment, the solution is suitable for storage for at least 1, 2, or 3 years at 2-8° C. (e.g., 4° C.).

As exemplified in the working examples described herein, the solutions described herein are suitable for maintaining an anti-C5 antibody at approximately 100 mg/mL in predominantly monomeric form for up to two years at approximately 2° C. to 8° C. As used herein, an anti-C5 antibody formulated at a high concentration in a featured aqueous solution is “predominantly monomeric,” or in “predominantly monomeric form,” if the antibody present in the solution is at least 95 (e.g., at least 95.1, 95.2, 95.3, 95.4, 95.5, 95.6, 95.7, 95.8, 95.9, 96, 96.1, 96.2, 96.3, 96.4, 96.5, 96.6, 96.7, 96.8, 96.9, 97, 97.1, 97.2, 97.3, 97.4, 97.5, 97.6, 97.7, 97.8, 97.9, 98, 98.1, 98.2, 98.3, 98.4, 98.5, 98.6, 98.7, 98.8, 98.9, 99, 99.1, 99.2, 99.3, 99.4, 99.5, 99.6, 99.7, 99.8, or 99.9 or greater) % monomeric, e.g., as determined using size exclusion chromatography high performance liquid chromatography (SEC-HPLC, such as gel permeation HPLC). In one embodiment, the anti-C5 antibody in the solutions described here can remain predominantly monomeric after storage for at least one month (e.g., at least two months, three months, four months, five months, six months, seven months, eight months, nine months, 10 months, 11 months, 12 months, 13 months, 14 months, 15 months, 16 months, 17 months, 18 months, 19 months, 20 months, 21 months, 22 months, 23 months, 24 months, or more) at approximately 2° C. to 8° C. (e.g., storage at, e.g., 1, 2, 3, 4, 5, 6, 7, 8, 9, or 10° C.).

In one embodiment of any of the solutions described herein, the anti-C5 antibody (e.g., ravulizumab) remains at least 95 (e.g., at least 96, 97, 98, or 99)% monomeric during storage at 2° C. to 8° C. for at least six months as determined by SEC-HPLC (e.g., gel permeation HPLC). In another embodiment, the anti-C5 antibody remains at least 95 (e.g., at least 96, 97, 98, or 99)% monomeric during storage at 2° C. to 8° C. for at least nine months as determined by SEC-HPLC. In another embodiment, the anti-C5 antibody remains at least 95 (e.g., at least 96, 97, 98, or 99)% monomeric during storage at 2° C. to 8° C. for at least one year as determined by SEC-HPLC. In another embodiment, the anti-C5 antibody remains at least 95 (e.g., at least 96, 97, 98, or 99)% monomeric during storage at 2° C. to 8° C. for at least 18 months as determined by SEC-HPLC. In another embodiment, the anti-C5 antibody remains at least 95 (e.g., at least 96, 97, 98, or 99)% monomeric during storage at 2° C. to 8° C. for at least two years as determined by SEC-HPLC.

In another embodiment, less than 5 (e.g., less than 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1) % of the antibody in the solution is oligomeric, aggregated, and/or fragmented. As used herein, antibody fragmentation refers to improperly assembled constituents or degradation products of a whole antibody having a lower molecular weight than the whole antibody. Such fragmentation forms include, but are not limited to, a free monomeric heavy chain polypeptide, a dimeric heavy chain polypeptide (e.g., disulfide-linked heavy chain polypeptide), a dimeric heavy chain polypeptide bound to one light chain polypeptide, a monomeric heavy chain polypeptide bound to one light chain polypeptide, or further degradation product(s) or fragment(s) of a light chain or heavy chain polypeptide. In some embodiments, less than 2 (e.g., less than 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1) % of the antibody is aggregated after storage for at least one month (e.g., at least two months, three months, four months, five months, six months, seven months, eight months, nine months, 10 months, 11 months, 12 months, 13 months, 14 months, 15 months, 16 months, 17 months, 18 months, 19 months, 20 months, 21 months, 22 months, 23 months, 24 months, or more) at 2° C. to 8° C. In some embodiments, less than 1 (e.g., less than 0.9, 0.8, 0.7, 0.6, 0.5, 0.4, 0.3, 0.2, or 0.1) % of the antibody is fragmented after storage for at least one month (e.g., at least two months, three months, four months, five months, six months, seven months, eight months, nine months, 10 months, 11 months, 12 months, 13 months, 14 months, 15 months, 16 months, 17 months, 18 months, 19 months, 20 months, 21 months, 22 months, 23 months, 24 months, or more) at 2° C. to 8° C. Methods for determining the amount of monomeric antibody, as well as the amount of oligomeric, aggregated, or fragmented forms of the anti-C5 antibody present in solution are described herein and exemplified in the working examples. For example, a skilled artisan can determine the percentage of whole, fragmented, unfolded intermediates, and/or aggregated antibody species present in a given solution using, e.g., size exclusion chromatography high-performance liquid chromatography (SEC-HPLC, such as gel permeation HPLC), static light scattering (SLS), Fourier transform infrared spectroscopy (FTIR), circular dichroism (CD), urea-induced protein unfolding techniques, intrinsic tryptophan fluorescence, non-reducing sodium dodecyl sulfate polyacrylamide gel electrophoresis (SDS-PAGE), and differential scanning calorimetry (DSC).

In one embodiment of any of the solutions described herein, less than 5% of the anti-C5 antibody (e.g., ravulizumab) in the solution is aggregated as determined by SEC-HPLC (e.g., gel permeation HPLC). In another embodiment, less than 4% of the anti-C5 antibody in the solution is aggregated as determined by SEC-HPLC. In another embodiment, less than 3% of the anti-C5 antibody in the solution is aggregated as determined by SEC-HPLC. In another embodiment, less than 2% of the anti-C5 antibody in the solution is aggregated as determined by SEC-HPLC. In another embodiment, less than 1% of the anti-C5 antibody in the solution is aggregated as determined by SEC-HPLC.

As described herein, the anti-C5 antibody containing solutions featured herein can retain at least 90 (e.g., 91, 92, 93, 94, 95, 96, 97, 98, 99, or even 100) % of their biological/functional activity (e.g., ability to bind to human C5) after storage for at least one month (e.g., at least two months, three months, four months, five months, six months, seven months, eight months, nine months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 36 months or more) at 2° C. to 8° C.

In another embodiment, anti-C5 antibody (e.g., ravulizumab) present in a solution described herein can retain, at least 90 (e.g., 91, 92, 93, 94, 95, 96, 97, 98, 99, or even 100) % of its activity to inhibit hemolysis after storage for at least one month (e.g., at least two months, three months, four months, five months, six months, seven months, eight months, nine months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 36 months or more at 2° C. to 8° C. Suitable hemolytic assay methods for determining whether an antibody in a featured solution retains its activity are described herein and known in the art, e.g., in vitro hemolytic assays using avian or porcine erythrocytes. Suitable methods for evaluating the ability of an antibody preparation to bind to human complement component C5 are known in the art and described herein.

In another embodiment of any of the solutions described herein, the anti-C5 antibody (e.g., ravulizumab) retains at least 80 (e.g., at least 81, 82, 83, 84, 85, 86, 87, 88, 89, 90, 91, 92, 93, 94, 95, 96, 97, 98, or 99)% of its C5-binding activity during storage at 2° C. to 8° C. for at least six months, as compared to a reference anti-C5 antibody corresponding to the anti-C5 antibody prior to storage. In another embodiment, the anti-C5 antibody (e.g., ravulizumab) retains at least 80 (e.g., at least 81, 82, 83, 84, 85, 86, 87, 88, 89, 90, 91, 92, 93, 94, 95, 96, 97, 98, or 99)% of its C5-binding activity during storage at 2° C. to 8° C. for at least nine months, as compared to a reference anti-C5 antibody corresponding to the anti-C5 antibody prior to storage. In another embodiment, the anti-C5 antibody (e.g., ravulizumab) retains at least 80 (e.g., at least 81, 82, 83, 84, 85, 86, 87, 88, 89, 90, 91, 92, 93, 94, 95, 96, 97, 98, or 99)% of its C5-binding activity during storage at 2° C. to 8° C. for at least one year, as compared to a reference anti-C5 antibody corresponding to the anti-C5 antibody prior to storage. In another embodiment, the anti-C5 antibody (e.g., ravulizumab) retains at least 80 (e.g., at least 81, 82, 83, 84, 85, 86, 87, 88, 89, 90, 91, 92, 93, 94, 95, 96, 97, 98, or 99)% of its C5-binding activity during storage at 2° C. to 8° C. for at least eighteen months, as compared to a reference anti-C5 antibody corresponding to the anti-C5 antibody prior to storage. In another embodiment, the anti-C5 antibody (e.g., ravulizumab) retains at least 80 (e.g., at least 81, 82, 83, 84, 85, 86, 87, 88, 89, 90, 91, 92, 93, 94, 95, 96, 97, 98, or 99)% of its C5-binding activity during storage at 2° C. to 8° C. for at least two years, as compared to a reference anti-C5 antibody corresponding to the anti-C5 antibody prior to storage. In another embodiment, the anti-C5 antibody (e.g., ravulizumab) retains at least 80 (e.g., at least 81, 82, 83, 84, 85, 86, 87, 88, 89, 90, 91, 92, 93, 94, 95, 96, 97, 98, or 99)% of its C5-binding activity during storage at 2° C. to 8° C. for at least three years, as compared to a reference anti-C5 antibody corresponding to the anti-C5 antibody prior to storage.

In another embodiment of any of the solutions described herein, the anti-C5 antibody (e.g., ravulizumab) retains at least 80 (e.g., at least 81, 82, 83, 84, 85, 86, 87, 88, 89, 90, 91, 92, 93, 94, 95, 96, 97, 98, or 99)% of its ability to inhibit hemolysis during storage at 2° C. to 8° C. for at least nine months, as compared to a reference anti-C5 antibody corresponding to the anti-C5 antibody prior to storage. In another embodiment of any of the solutions described herein, the anti-C5 antibody (e.g., ravulizumab) retains at least 80 (e.g., at least 81, 82, 83, 84, 85, 86, 87, 88, 89, 90, 91, 92, 93, 94, 95, 96, 97, 98, or 99)% of its ability to inhibit hemolysis during storage at 2° C. to 8° C. for at least six months, as compared to a reference anti-C5 antibody corresponding to the anti-C5 antibody prior to storage. In another embodiment of any of the solutions described herein, the anti-C5 antibody (e.g., ravulizumab) retains at least 80 (e.g., at least 81, 82, 83, 84, 85, 86, 87, 88, 89, 90, 91, 92, 93, 94, 95, 96, 97, 98, or 99)% of its ability to inhibit hemolysis during storage at 2° C. to 8° C. for at least one year, as compared to a reference anti-C5 antibody corresponding to the anti-C5 antibody prior to storage. In another embodiment of any of the solutions described herein, the anti-C5 antibody (e.g., ravulizumab) retains at least 80 (e.g., at least 81, 82, 83, 84, 85, 86, 87, 88, 89, 90, 91, 92, 93, 94, 95, 96, 97, 98, or 99)% of its ability to inhibit hemolysis during storage at 2° C. to 8° C. for at least 18 months, as compared to a reference anti-C5 antibody corresponding to the anti-C5 antibody prior to storage. In another embodiment of any of the solutions described herein, the anti-C5 antibody (e.g., ravulizumab) retains at least 80 (e.g., at least 81, 82, 83, 84, 85, 86, 87, 88, 89, 90, 91, 92, 93, 94, 95, 96, 97, 98, or 99)% of its ability to inhibit hemolysis during storage at 2° C. to 8° C. for at least two years, as compared to a reference anti-C5 antibody corresponding to the anti-C5 antibody prior to storage. In another embodiment of any of the solutions described herein, the anti-C5 antibody (e.g., ravulizumab) retains at least 80 (e.g., at least 81, 82, 83, 84, 85, 86, 87, 88, 89, 90, 91, 92, 93, 94, 95, 96, 97, 98, or 99)% of its ability to inhibit hemolysis during storage at 2° C. to 8° C. for at least three years, as compared to a reference anti-C5 antibody corresponding to the anti-C5 antibody prior to storage.

The aqueous solutions described herein can contain one or more common agents (e.g., one or more excipients and/or additives, such as buffering agents, sugars or saccharides, salts, surfactants, solubilizers, diluents, binders, stabilizers, salts, lipophilic solvents, amino acids, chelators, and/or preservatives).

In one embodiment, the aqueous solution contains one or more buffering agents. As used herein, the term “buffering agent” refers to one or more components that when added to an aqueous solution is able to protect the solution against variations in pH when adding acid or alkali, or upon dilution with a solvent. In one embodiment, the solution comprises at least one or more buffering agents. Non-limiting examples of typical buffers that can be included in the wash solution(s) include Tris (tris(hydroxymethyl)methylamine), bis-Tris, bis-Tris propane, histidine, triethanolamine, diethanolamine, formate, acetate, MES (2-(N-morpholino)ethanesulfonic acid), phosphate, HEPES (4-2-hydroxyethyl-1-piperazineethanesulfonic acid), citrate, MOPS (3-(N-morpholino)propanesulfonic acid), TAPS (3{[tris(hydroxymethyl)methyl]amino}propanesulfonic acid), Bicine (N,N-bis(2-hydroxyethyl)glycine), Tricine (N-tris(hydroxymethyl)methylglycine), TES (2-{[tris(hydroxymethyl)methyl]amino}ethanesulfonic acid), PIPES (piperazine-N,N′-bis(2-ethanesulfonic acid), cacodylate (dimethylarsinic acid) SSC (saline sodium citrate), and sodium phosphate.

In another embodiment, the buffering agent is an amino acid. The amino acid can be, e.g., one selected from the group consisting of histidine (e.g., L-histidine), serine (e.g., L-serine), and glycine (e.g., L-glycine). In another embodiment, the solution comprises two or more buffering agents.

In one embodiment, the featured solutions do not include a free amino acid as a buffering agent. In another embodiment, the featured solutions include but one free amino acid (e.g., histidine) as a buffering agent. In another embodiment, the featured solutions can include two or more (e.g., two, three, four, five, six, or seven or more) different amino acids as buffering agents, e.g., serine and histidine.

The concentration of the buffer is sufficient to maintain the desired pH and may also be varied, for example, to maintain the isotonicity of the formulation. Typical concentrations of conventional buffering agents employed in parenteral formulations can be found in: Pharmaceutical Dosage Form: Parenteral Medications, Volume 1, 2nd Edition, Chapter 5, p. 194, De Luca and Boylan, “Formulation of Small Volume Parenterals”, Table 5: Commonly used additives in Parenteral Product. In one embodiment, the concentration of the one or more buffering agents in the formulation is about 10 mM to 300 mM, inclusive. In another embodiment, the solution comprises at least one buffering agent at a concentration of 10 mM to 200 mM, inclusive. In another embodiment, the aqueous solution described herein contains a buffering agent at a concentration of at least 10 (e.g., at least 15, 20, 25, 30, 35, 40, 50, 60, 70, 80, 90, 100, 110, 120, 130, 140, 150, 160, 170, 180, 190, 200, 210, 220, 230, 240, 250, 260, 270, 280, 290, or 300 or more) mM. In another embodiment, the aqueous solution includes a buffering agent at a concentration of between about 10 mM to 50 mM, 15 mM to 50 mM, 20 mM to 50 mM, 25 mM to 50 mM, 30 mM to 50 mM, 40 mM to 50 mM, 10 mM to 100 mM, 15 mM to 100 mM, 20 mM to 100 mM, 25 mM to 100 mM, 30 mM to 100 mM, 40 mM to 100 mM, 10 mM to 150 mM, 15 mM to 150 mM, 20 mM to 150 mM, 25 mM to 150 mM, 30 mM to 150 mM, 40 mM to 150 mM, 50 mM to 100 mM, 60 mM to 100 mM, 70 mM to 100 mM, 80 mM to 100 mM, 50 mM to 150 mM, 60 mM to 150 mM, 70 mM to 150 mM, 80 mM to 150 mM, 90 mM to 150 mM, 100 mM to 150 mM, 10 mM to 200 mM, 15 mM to 200 mM, 20 mM to 200 mM, 25 mM to 200 mM, 30 mM to 200 mM, 40 mM to 200 mM, 50 mM to 200 mM, 60 mM to 200 mM, 70 mM to 200 mM, 80 mM to 200 mM, 90 mM to 200 mM, 100 mM to 200 mM, 150 mM to 200 mM, 10 mM to 250 mM, 15 mM to 250 mM, 20 mM to 250 mM, 25 mM to 250 mM, 30 mM to 250 mM, 40 mM to 250 mM, 50 mM to 250 mM, 60 mM to 250 mM, 70 mM to 250 mM, 80 mM to 250 mM, 90 mM to 250 mM, 100 mM to 250 mM, 150 mM to 250 mM, or 200 mM to 250 mM. In another embodiment, the concentration of the buffer in the formulation is about 20 mM, 25 mM, 30 mM, 35 mM, 40 mM, 45 mM, 50 mM, 55 mM, 60 mM, 65 mM, 70 mM, 75 mM, 80 mM, 90 mM, 95 mM or about 100 mM. In another embodiment, the buffering agent is present in the solution at a concentration of at least, or equal to, 20 mM. In another embodiment, buffering agent is present in the solution at a concentration of at least, or equal to, 25 mM. In another embodiment, buffering agent is present in the solution at a concentration of at least, or equal to, 50 mM. In embodiments where a featured solution contains two or more (e.g., at least two, three, four, five, six, seven, eight, nine, or 10 or more) different buffering agents, each of the two or more buffering agents can independently be present at, e.g., one of the above described concentrations.

In a particular embodiment, the buffering agent is sodium phosphate monobasic. In another particular embodiment, the buffering agent is sodium phosphate dibasic. In another particular embodiment, the buffering agent is sodium phosphate monobasic at a concentration of about 33.1 mM. In another particular embodiment, the buffering agent is sodium phosphate dibasic at a concentration of about 16.5 mM.

In one embodiment, the solution contains one or more surfactants, such as an anionic, cationic, or nonionic surfactant. As used herein, the term “surfactant” refers to a surface active molecule containing both a hydrophobic portion (e.g., alkyl chain) and a hydrophilic portion (e.g., carboxyl and carboxylate groups). In one embodiment, the surfactant in the formulation is a non-ionic surfactant. In certain embodiments, the surfactant in the formulation is a polyoxyethylene sorbitan fatty acid ester, for example, polysorbate 20, 40, 60, 80, or a combination of one or more thereof. In one embodiment, the surfactant in the formulation is polysorbate 80 (Tween 80). The amount of surfactant added to the formulation is sufficient to reduce aggregation of the formulated antibody and/or minimize the formation of particulates in the formulation. For example, the surfactant may be present in the formulation in an amount from about 0.001% to about 1%, or about 0.001% to about 0.5%, or about 0.01% to about 0.2%. In one embodiment, the aqueous solutions contain a surfactant at a concentration of at least, or approximately, 0.001 (e.g., at least, or approximately, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 or more) %. In another embodiment, the aqueous solution contains no more than 0.2 (e.g., no more than 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01, 0.009, 0.008, 0.007, 0.006, 0.005, 0.004, 0.003, 0.002, or 0.001) % of a pharmaceutically-acceptable surfactant.

In another embodiment, the formulations comprise polysorbate at a concentration from about 0.001% to about 0.5%, from about 0.005% to about 0.2%, from about 0.01% to about 0.1%, or from about 0.02% to about 0.06%, or about 0.03% to about 0.05% (w/v). In certain embodiments, the formulation comprises a polysorbate at a concentration of 0.01%, or 0.02%, or 0.03%, or 0.04%, or 0.05%, or 0.06%, or 0.07%, or 0.08%, or 0.09%, or 0.1%, or 0.15%, or 0.2% (w/v). In certain embodiments, the surfactant is present in the formulation in an amount of 0.02% or about 0.04% (w/v). In one embodiment, the surfactant is present in the formulation in an amount of 0.05% (w/v).

In one embodiment, the formulation comprises at least about 0.01%, at least about 0.02%, at least about 0.05%, at least about 0.1%, at least about 0.2%, at least about 0.3%, at least about 0.4%, or at least about 0.5% polysorbate 80. In certain embodiment, the formulation comprises between about 0.01% and about 0.5%, between about 0.01% and about 0.3%, between about 0.001% and about 0.2%, between about 0.02% and about 0.5%, between about 0.02% and about 0.3%, between about 0.02% and about 0.2%, between about 0.05% and about 0.5%, between about 0.05% and about 0.3%, between about 0.05% and about 0.2%, between about 0.075% and about 0.5%, between about 0.075% and about 0.3%, or between about 0.075% and about 0.2% polysorbate 80. In a further embodiment, the formulation comprises about 0.01%, about 0.02%, about 0.05%, about 0.1%, about 0.2%, about 0.3%, about 0.4%, or about 0.5% polysorbate 80. In one embodiment, the formulation comprises about 0.05% polysorbate 80. In one embodiment, the formulation comprises about comprises about 0.04% polysorbate 80. In one embodiment, the formulation comprises about 0.03% polysorbate 80. In one embodiment, the formulation comprises about 0.02% polysorbate 80. In one embodiment, the formulation comprises about 0.01% polysorbate 80.

In one embodiment, the aqueous solution contains one or more salts, e.g., sodium chloride, potassium chloride, or magnesium chloride. In some embodiments, an aqueous solution described herein contains a salt at a concentration of at least 10 (e.g., at least 15, 20, 25, 30, 35, 40, 50, 60, 70, 80, 90, 100, 110, 120, 130, 140, 150, 160, 170, 180, 190, 200, 210, 220, 230, 240, 250, 260, 270, 280, 290, or 300 or more) mM. In some embodiments, an aqueous solution described herein can include a salt at a concentration of less than, or approximately, 200 (e.g., less than, or approximately, 190, 180, 170, 160, 150, 140, 130, 120, 110, 100, 90, 80, 70, 60, 50, 40, 30, 25, 20, 15, or 10) mM. In some embodiments, an aqueous solution described herein can include a salt at a concentration of between about 10 mM to 50 mM, 15 mM to 50 mM, 20 mM to 50 mM, 25 mM to 50 mM, 30 mM to 50 mM, 40 mM to 50 mM, 10 mM to 100 mM, 15 mM to 100 mM, 20 mM to 100 mM, 25 mM to 100 mM, 30 mM to 100 mM, 40 mM to 100 mM, 10 mM to 150 mM, 15 mM to 150 mM, 20 mM to 150 mM, 25 mM to 150 mM, 30 mM to 150 mM, 40 mM to 150 mM, 50 mM to 100 mM, 60 mM to 100 mM, 70 mM to 100 mM, 80 mM to 100 mM, 50 mM to 150 mM, 60 mM to 150 mM, 70 mM to 150 mM, 80 mM to 150 mM, 90 mM to 150 mM, 100 mM to 150 mM, 10 mM to 200 mM, 15 mM to 200 mM, 20 mM to 200 mM, 25 mM to 200 mM, 30 mM to 200 mM, 40 mM to 200 mM, 50 mM to 200 mM, 60 mM to 200 mM, 70 mM to 200 mM, 80 mM to 200 mM, 90 mM to 200 mM, 100 mM to 200 mM, 150 mM to 200 mM, 10 mM to 250 mM, 15 mM to 250 mM, 20 mM to 250 mM, 25 mM to 250 mM, 30 mM to 250 mM, 40 mM to 250 mM, 50 mM to 250 mM, 60 mM to 250 mM, 70 mM to 250 mM, 80 mM to 250 mM, 90 mM to 250 mM, 100 mM to 250 mM, 150 mM to 250 mM, or 200 mM to 250 mM. In embodiments where a featured solution contains two or more (e.g., at least two, three, four, five, six, seven, eight, nine, or 10 or more) different salts, each of the two or more salts can independently be present at, e.g., one of the above described concentrations. In a particular embodiment, the aqueous solution contains sodium chloride.

In one embodiment, the aqueous solution contains one or more carbohydrate excipients. Suitable carbohydrate excipients are described in, e.g., Katakam and Banga (1995) J Pharm Pharmacol 47(2):103-107; Andya et al. (2003) AAPS PharmSci 5(2): Article 10; and Shire (2009) “Current Trends in Monoclonal Antibody Development and Manufacturing,” Volume 11, Springer, 354 pages. Carbohydrate excipients suitable for use in the solutions described herein include, without limitation, monosaccharides such as fructose, maltose, galactose, glucose, D-mannose, and sorbose; disaccharides such as lactose, sucrose, trehalose, and cellobiose; polysaccharides such as maltodextrins, dextrans, and starches; and sugar alcohols such as mannitol, xylitol, maltitol, lactitol, and sorbitol. In one embodiment, a carbohydrate excipient is present in a solution featured herein at a concentration of at least, or approximately, 0.5 (e.g., at least, or approximately, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.25, 3.5, 3.75, 4, 4.25, 4.5, 4.75, 5, 5.5, 6, 6.5, 7, 7.5, 8, 8.5, 9, 9.5, 10, or more) %. In embodiments where a featured solution contains two or more (e.g., at least two, three, four, five, six, seven, eight, nine, or 10 or more) different carbohydrate excipients, each excipient can, independently, be present at any of the above-described concentrations.

In another embodiment, the stable aqueous solution comprises one or more stabilizing agents.

Exemplary stabilizers include, but are not limited to polyols, sugars (e.g., sucrose or trehalose), amino acids (e.g., arginine), amines, and salting out salts. In one embodiment, the solution comprises at least one stabilizing agent at a concentration of 2-10%, inclusive. In another embodiment, the solution comprises at least one or more stabilizing agents at a concentration of 10 mM to 50 mM, inclusive. In another embodiment, the stabilizing agent is present in the solution at a concentration of at least, or equal to, 20 mM. In another embodiment, the stabilizing agent is present in the solution at a concentration of at least, or equal to, 25 mM. In another embodiment, the stabilizing agent is present in the solution at a concentration of at least, or equal to, 50 mM. In another embodiment, the solution comprises 5% sucrose. In another embodiment, the solution comprises between about 30 mg/mL to 60 mg/mL of sucrose (e.g., about 30 mg/mL, 31 mg/mL, 32 mg/mL, 33 mg/mL, 34 mg/ml, 35 mg/ml, 36 mg/mL, 37 mg/mL, 38 mg/mL, 39 mg/mL, 40 mg/mL, 41 mg/mL, 42 mg/mL, 43 mg/mL, 44 mg/mL, 45 mg/mL, 46 mg/mL, 47 mg/mL, 48 mg/mL, 49 mg/mL, 50 mg/mL, 51 mg/mL, 52 mg/mL, 53 mg/mL, 54 mg/mL, 55 mg/mL, 56 mg/mL, 57 mg/mL, 58 mg/mL, 59 mg/mL, or 60 mg/mL). In a particular embodiment, the solution comprises 50 mg/mL of sucrose. In another embodiment, the solution comprises between 20 to 30 mM arginine (e.g., L-arginine). For example, in one embodiment, the solution comprises 20 mM arginine, 21 mM arginine, 22 mM arginine, 23 mM arginine, 24 mM arginine, 25 mM arginine, 26 mM arginine, 27 mM arginine, 28 mM arginine, 29 mM arginine, or 30 mM arginine. In a particular embodiment, the solution comprises 25 mM L-arginine.

In another embodiment, the solution comprises water for injection.

In another embodiment, the pH is 7.0, 7.1, 7.2, 7.3, 7.4, 7.5, 7.6, 7.7, 7.8, or 7.9. In another embodiment, the pH of the solution is between 7.0 and 7.4. In another embodiment, the pH of the solution is between 7.2 and 7.8. In another embodiment, the pH of the solution is between 7.2 and 7.6. In a particular embodiment, the pH of the solution is 7.4.

In another embodiment, the solution is in any container suitable for storage of medicines and other therapeutic compositions. For example, the solution may be contained within a sealed and sterilized plastic or glass container having a defined volume, such as a vial, ampule, syringe, cartridge, or bottle. Different types of vials can be used to contain the solutions of the present invention including, e.g., glass or plastic vials. In one embodiment, the vial is a 3 mL single use vial. In another embodiment, the vial is a 3 mL single use vial comprising 300 mg of ravulizumab. In another embodiment, the vial is an 11 mL single use vial. In another embodiment, the vial is an 11 mL single use vial comprising 1,100 mg of ravulizumab.

In another embodiment, the solution is diluted prior to administration. For example, in one embodiment, the solution is diluted prior to administration with normal saline (0.9% sodium chloride). In another embodiment, the solution is diluted prior to administration to a concentration of 50±15 mg/mL ravulizumab (e.g., 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65 mg/mL ravulizumab). In a particular embodiment, the solution is diluted prior to administration to a concentration of about 50 mg/mL ravulizumab. In another embodiment, the solution is stable after dilution for up to 24 hours at 2° C.-8° C. (36° F.-46° F.). In another embodiment, the amount of saline (e.g., sodium chloride) can be optionally decreased, for example, when the solution is administered to a patient on a sodium-controlled (e.g., low sodium) diet.

In one embodiment, the stable aqueous solution comprises no more than seven agents in addition to the anti-C5 antibody, e.g., ravulizumab. In another embodiment, the stable aqueous solution comprises no more than six agents in addition to the anti-C5 antibody, e.g., ravulizumab. In another embodiment, the stable aqueous solution comprises no more than five agents in addition to the anti-C5 antibody, e.g., ravulizumab. In another embodiment, the stable aqueous solution comprises no more than four agents in addition to the anti-C5 antibody, e.g., ravulizumab. In another embodiment, the stable aqueous solution comprises no more than three agents in addition to the anti-C5 antibody, e.g., ravulizumab. In another embodiment, the stable aqueous solution comprises no more than two agents in addition to the anti-C5 antibody, e.g., ravulizumab. In another embodiment, the stable aqueous solution comprises no more than one agent in addition to the anti-C5 antibody, e.g., ravulizumab.

In another embodiment, the stable aqueous solution comprises or consists of ravulizumab at a concentration of 100±20 (e.g., 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120) mg/mL; 50±15 (e.g., 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65) mM phosphate buffer; 5±3 (e.g., 2, 3, 4, 5, 6, 7, or 8) % sucrose; and 25±10 (e.g., 15, 16, 17, 18, 19, 20, 21, 22, 23, 24, 25, 26, 27, 28, 29, 30, 31, 32, 33, 34, or 35) mM arginine; wherein the solution has a pH of 7.4±0.5 (e.g., 6.9, 7, 7.1, 7.2, 7.3, 7.4, 7.5, 7.6, 7.7, 7.8, or 7.9).

In one embodiment, the stable aqueous solution comprises: (a) ravulizumab at a concentration of about 100 mg/mL; (b) about 50 mM sodium phosphate (c) about 0.05% w/v polysorbate 80; (d) about 25 mM L-arginine; (e) about 50 mg/mL sucrose; and (f) water for injection; wherein the solution has a pH of about 7.4 and is contained in a 3 mL single use vial comprising 300 mg of ravulizumab.

In another embodiment, the stable aqueous solution consists of: (a) ravulizumab at a concentration of about 100 mg/mL; (b) about 50 mM sodium phosphate (c) about 0.05% w/v polysorbate 80; (d) about 25 mM L-arginine; (e) about 50 mg/mL sucrose; and (f) water for injection; wherein the solution has a pH of about 7.4 and is contained in a 3 mL single use vial comprising 300 mg of ravulizumab.

In one embodiment, the stable aqueous solution consists of: (a) ravulizumab at a concentration of 100 mg/mL; (b) about 50 mM sodium phosphate (c) 0.05% w/v polysorbate 80; (d) 25 mM L-arginine; (e) 50 mg/mL sucrose; and (f) water for injection; wherein the solution has a pH of about 7.4 and is contained in a 3 mL single use vial comprising 300 mg of ravulizumab.

In one embodiment, the stable aqueous solution comprises: (a) ravulizumab at a concentration of about 100 mg/mL; (b) about 50 mM sodium phosphate (c) about 0.05% w/v polysorbate 80; (d) about 25 mM L-arginine; (e) about 5% w/v sucrose; and (f) water for injection; wherein the solution has a pH of about 7.4 and is contained in a 3 mL single use vial comprising 300 mg of ravulizumab.

In another embodiment, the stable aqueous solution consists of: (a) ravulizumab at a concentration of about 100 mg/mL; (b) about 50 mM sodium phosphate (c) about 0.05% w/v polysorbate 80; (d) about 25 mM L-arginine; (e) about 5% w/v sucrose; and (f) water for injection; wherein the solution has a pH of about 7.4 and is contained in a 3 mL single use vial comprising 300 mg of ravulizumab.

In one embodiment, the stable aqueous solution consists of: (a) ravulizumab at a concentration of 100 mg/mL; (b) about 50 mM sodium phosphate (c) 0.05% w/v polysorbate 80; (d) 25 mM L-arginine; (e) 5% w/v sucrose; and (f) water for injection; wherein the solution has a pH of about 7.4 and is contained in a 3 mL single use vial comprising 300 mg of ravulizumab.

In another embodiment, the stable aqueous solution comprises: (a) ravulizumab at a concentration of about 100 mg/mL; (b) about 33.1 mM sodium phosphate, monobasic; (c) about 16.5 mM sodium phosphate, dibasic; (d) about 0.05% w/v polysorbate 80; (f) about 25 mM L-arginine; (g) about 5% w/v sucrose; and (h) water for injection; wherein the solution has a pH of about 7.4 and is contained in a 3 mL single use vial comprising 300 mg of ravulizumab.

In another embodiment, the stable aqueous solution consists of: (a) ravulizumab at a concentration of about 100 mg/mL; (b) about 33.1 mM sodium phosphate, monobasic; (c) about 16.5 mM sodium phosphate, dibasic; (d) about 0.05% w/v polysorbate 80; (f) about 25 mM L-arginine; (g) about 5% w/v sucrose; and (h) water for injection; wherein the solution has a pH of about 7.4 and is contained in a 3 mL single use vial comprising 300 mg of ravulizumab.

In another embodiment, the stable aqueous solution consists of: (a) ravulizumab at a concentration of 100 mg/mL; (b) 33.1 mM sodium phosphate, monobasic; (c) 16.5 mM sodium phosphate, dibasic; (d) 0.05% w/v polysorbate 80; (f) 25 mM L-arginine; (g) 5% w/v sucrose; and (h) water for injection; wherein the solution has a pH of 7.4 and is contained in a 3 mL single use vial comprising 300 mg of ravulizumab. In another embodiment, the stable aqueous solution comprises: (a) ravulizumab at a concentration of about 100 mg/mL; (b) about 33.1 mM sodium phosphate, monobasic; (c) about 16.5 mM sodium phosphate, dibasic; (d) about 0.05% w/v polysorbate 80; (f) about 25 mM L-arginine; (g) about 50 mg/mL sucrose; and (h) water for injection; wherein the solution has a pH of about 7.4 and is contained in a 3 mL single use vial comprising 300 mg of ravulizumab.

In another embodiment, the stable aqueous solution consists of: (a) ravulizumab at a concentration of about 100 mg/mL; (b) about 33.1 mM sodium phosphate, monobasic; (c) about 16.5 mM sodium phosphate, dibasic; (d) about 0.05% w/v polysorbate 80; (f) about 25 mM L-arginine; (g) about 50 mg/mL sucrose; and (h) water for injection; wherein the solution has a pH of about 7.4 and is contained in a 3 mL single use vial comprising 300 mg of ravulizumab.

In another embodiment, the stable aqueous solution consists of: (a) ravulizumab at a concentration of 100 mg/mL; (b) 33.1 mM sodium phosphate, monobasic; (c) 16.5 mM sodium phosphate, dibasic; (d) 0.05% w/v polysorbate 80; (f) 25 mM L-arginine; (g) 50 mg/mL sucrose; and (h) water for injection; wherein the solution has a pH of 7.4 and is contained in a 3 mL single use vial comprising 300 mg of ravulizumab.

In another embodiment, the stable aqueous solution comprises: (a) ravulizumab at a concentration of about 100 mg/mL; (b) about 50 mM sodium phosphate (c) about 0.05% w/v polysorbate 80; (d) about 25 mM L-arginine; (e) about 50 mg/mL sucrose; and (f) water for injection, wherein the solution has a pH of about 7.4 and is contained in a 11 mL single use vial comprising 1100 mg of ravulizumab.

In another embodiment, the stable aqueous solution consists of: (a) ravulizumab at a concentration of about 100 mg/mL; (b) about 50 mM sodium phosphate (c) about 0.05% w/v polysorbate 80; (d) about 25 mM L-arginine; (e) about 50 mg/mL sucrose; and (f) water for injection, wherein the solution has a pH of about 7.4 and is contained in a 11 mL single use vial comprising 1100 mg of ravulizumab.

In another embodiment, the stable aqueous solution consists of: (a) ravulizumab at a concentration of 100 mg/mL; (b) about 50 mM sodium phosphate (c) 0.05% w/v polysorbate 80; (d) 25 mM L-arginine; (e) 50 mg/mL sucrose; and (f) water for injection, wherein the solution has a pH of about 7.4 and is contained in a 11 mL single use vial comprising 1100 mg of ravulizumab.

In another embodiment, the stable aqueous solution comprises: (a) ravulizumab at a concentration of about 100 mg/mL; (b) about 50 mM sodium phosphate (c) about 0.05% w/v polysorbate 80; (d) about 25 mM L-arginine; (e) about 5% w/v sucrose; and (f) water for injection, wherein the solution has a pH of about 7.4 and is contained in a 11 mL single use vial comprising 1100 mg of ravulizumab.

In another embodiment, the stable aqueous solution consists of: (a) ravulizumab at a concentration of about 100 mg/mL; (b) about 50 mM sodium phosphate (c) about 0.05% w/v polysorbate 80; (d) about 25 mM L-arginine; (e) about 5% w/v sucrose; and (f) water for injection, wherein the solution has a pH of about 7.4 and is contained in a 11 mL single use vial comprising 1100 mg of ravulizumab.

In another embodiment, the stable aqueous solution consists of: (a) ravulizumab at a concentration of 100 mg/mL; (b) about 50 mM sodium phosphate (c) 0.05% w/v polysorbate 80; (d) 25 mM L-arginine; (e) 5% w/v sucrose; and (f) water for injection, wherein the solution has a pH of about 7.4 and is contained in a 11 mL single use vial comprising 1100 mg of ravulizumab.

In another embodiment, the stable aqueous solution comprises: (a) ravulizumab at a concentration of about 100 mg/mL; (b) about 33.1 mM sodium phosphate, monobasic; (c) about 16.5 mM sodium phosphate, dibasic; (d) about 0.05% w/v polysorbate 80; (f) about 25 mM L-arginine; (g) about 5% w/v sucrose; and (h) water for injection, wherein the solution has a pH of about 7.4 and is contained in a 11 mL single use vial comprising 1100 mg of ravulizumab.

In another embodiment, the stable aqueous solution consists of: (a) ravulizumab at a concentration of about 100 mg/mL; (b) about 33.1 mM sodium phosphate, monobasic; (c) about 16.5 mM sodium phosphate, dibasic; (d) about 0.05% w/v polysorbate 80; (f) about 25 mM L-arginine; (g) about 5% w/v sucrose; and (h) water for injection, wherein the solution has a pH of about 7.4 and is contained in a 11 mL single use vial comprising 1100 mg of ravulizumab.

In another embodiment, the stable aqueous solution consists of: (a) ravulizumab at a concentration of 100 mg/mL; (b) 33.1 mM sodium phosphate, monobasic; (c) 16.5 mM sodium phosphate, dibasic; (d) 0.05% w/v polysorbate 80; (f) 25 mM L-arginine; (g) 5% w/v sucrose; and (h) water for injection; wherein the solution has a pH of 7.4 and is contained in a 11 mL single use vial comprising 1100 mg of ravulizumab. In another embodiment, the stable aqueous solution comprises: (a) ravulizumab at a concentration of about 100 mg/mL; (b) about 33.1 mM sodium phosphate, monobasic; (c) about 16.5 mM sodium phosphate, dibasic; (d) about 0.05% w/v polysorbate 80; (f) about 25 mM L-arginine; (g) about 50 mg/mL sucrose; and (h) water for injection, wherein the solution has a pH of about 7.4 and is contained in a 11 mL single use vial comprising 1100 mg of ravulizumab.

In another embodiment, the stable aqueous solution consists of: (a) ravulizumab at a concentration of about 100 mg/mL; (b) about 33.1 mM sodium phosphate, monobasic; (c) about 16.5 mM sodium phosphate, dibasic; (d) about 0.05% w/v polysorbate 80; (f) about 25 mM L-arginine; (g) about 50 mg/mL sucrose; and (h) water for injection, wherein the solution has a pH of about 7.4 and is contained in a 11 mL single use vial comprising 1100 mg of ravulizumab.

In another embodiment, the stable aqueous solution consists of: (a) ravulizumab at a concentration of 100 mg/mL; (b) 33.1 mM sodium phosphate, monobasic; (c) 16.5 mM sodium phosphate, dibasic; (d) 0.05% w/v polysorbate 80; (f) 25 mM L-arginine; (g) 50 mg/mL sucrose; and (h) water for injection; wherein the solution has a pH of 7.4 and is contained in a 11 mL single use vial comprising 1100 mg of ravulizumab.

In another embodiment, the stable aqueous solution is sterile. In another embodiment, the solution is a sterile, translucent, clear to yellowish color, preservative-free solution. In another embodiment, the solution has a shelf-life of 18 months.

The solutions described herein can be formulated for any suitable mode of administration. In one embodiment, the solution is formulated for administration by a parenteral mode (e.g., intravenous, subcutaneous, intraperitoneal, or intramuscular injection). In a particular embodiment, the solution is formulated for subcutaneous administration. For example, in one embodiment, the stable aqueous solution comprises an anti-C5 antibody at a concentration of 100 mg/mL and is formulated for subcutaneous administration. In another particular embodiment, the solution is formulated for intravenous administration (e.g., via intravenous infusion). For example, in one embodiment, the stable aqueous solution comprises ravulizumab at a concentration of 100 mg/mL and is formulated for intravenous infusion.

### III. Methods of Treatment

The solutions described can be used to treat a variety of diseases and conditions in a human patient. In one embodiment, the solutions can be used to treat a complement-associated disorder, including, but not limited to: rheumatoid arthritis (RA); antiphospholipid antibody syndrome; lupus nephritis; ischemia-reperfusion injury; thrombotic microangiopathy (TMA), atypical hemolytic uremic syndrome (aHUS); typical or infectious hemolytic uremic syndrome (tHUS); dense deposit disease (DDD); paroxysmal nocturnal hemoglobinuria (PNH); neuromyelitis optica (NMO); multifocal motor neuropathy (MMN); multiple sclerosis (MS); macular degeneration (e.g., age-related macular degeneration (AMD)); hemolysis, elevated liver enzymes, and low platelets (HELLP) syndrome; thrombotic thrombocytopenic purpura (TTP); spontaneous fetal loss; Pauci-immune vasculitis; epidermolysis bullosa; recurrent fetal loss; and traumatic brain injury (see, e.g., Holers (2008) Immunological Reviews 223:300-316 and Holers and Thurman (2004) Molecular Immunology 41:147-152.)

In another embodiment, the complement-associated disorder is a complement-associated vascular disorder such as, but not limited to, a diabetes-associated vascular disorder (e.g., of the eye), central retinal vein occlusion, a cardiovascular disorder, myocarditis, a cerebrovascular disorder, a peripheral (e.g., musculoskeletal) vascular disorder, a renovascular disorder, a mesenteric/enteric vascular disorder, revascularization to transplants and/or replants, vasculitis, Henoch-Schönlein purpura nephritis, systemic lupus erythematosus-associated vasculitis, vasculitis associated with rheumatoid arthritis, immune complex vasculitis, Takayasu's disease, dilated cardiomyopathy, diabetic angiopathy, Kawasaki's disease (arteritis), venous gas embolus (VGE), and restenosis following stent placement, rotational atherectomy, and percutaneous transluminal coronary angioplasty (PTCA) (see, e.g., U.S. patent application publication no. 20070172483.)

Additional complement-associated disorders include, without limitation, myasthenia gravis, cold agglutinin disease, dermatomyositis, Graves' disease, atherosclerosis, Alzheimer's disease, Guillain-Barré Syndrome, Degos' disease, graft rejection (e.g., transplant rejection), sepsis, burn (e.g., severe burn), systemic inflammatory response sepsis, septic shock, spinal cord injury, glomerulonephritis, Hashimoto's thyroiditis, type I diabetes, psoriasis, pemphigus, autoimmune hemolytic anemia (AIHA), idiopathic thrombocytopenic purpura (ITP), Goodpasture syndrome, antiphospholipid syndrome (APS), catastrophic APS (CAPS), amyotrophic lateral sclerosis (ALS), Alzheimer's disease, and chronic inflammatory demyelinating neuropathy.

In another embodiment, the solutions described herein can be used to treat thrombotic microangiopathy (TMA), e.g., TMA associated with a complement-associated disorder such as any of the complement-associated disorders described herein, including atypical Hemolytic Uremic Syndrome (aHUS).

Complement-associated disorders also include complement-associated pulmonary disorders such as, but not limited to, asthma, bronchitis, a chronic obstructive pulmonary disease (COPD), an interstitial lung disease, α-1 anti-trypsin deficiency, emphysema, bronchiectasis, bronchiolitis obliterans, alveolitis, sarcoidosis, pulmonary fibrosis, and collagen vascular disorders.

In another embodiment, a solution described herein is administered to a subject to treat, prevent, or ameliorate at least one symptom of a complement-associated inflammatory response (e.g., the complement-associated inflammatory response aspect of a complement-associated disorder) in a subject. For example, a composition can be used to treat, prevent, and/or ameliorate one or more symptoms associated with a complement-associated inflammatory response such as graft rejection/graft-versus-host disease (GVHD), reperfusion injuries (e.g., following cardiopulmonary bypass or a tissue transplant), and tissue damage following other forms of traumatic injury such as a burn (e.g., a severe burn), blunt trauma, spinal injury, or frostbite. See, e.g., Park et al. (1999) Anesth Analg 99(1):42-48; Tofukuji et al. (1998) J Thorac Cardiovasc Surg 116(6):1060-1068; Schmid et al. (1997) Shock 8(2):119-124; and Bless et al. (1999) Am J Physiol 276(1):L57-L63.

In another embodiment, the complement-mediated disorder is a complement-mediated vascular disorder such as, but not limited to, a cardiovascular disorder, myocarditis, a cerebrovascular disorder, a peripheral (e.g., musculoskeletal) vascular disorder, a renovascular disorder, a mesenteric/enteric vascular disorder, revascularization to transplants and/or replants, vasculitis, Henoch-Schönlein purpura nephritis, systemic lupus erythematosus-associated vasculitis, vasculitis associated with rheumatoid arthritis, immune complex vasculitis, organ or tissue transplantation, Takayasu's disease, capillary leak syndrome, dilated cardiomyopathy, diabetic angiopathy, thoracic-abdominal aortic aneurysm, Kawasaki's disease (arteritis), venous gas embolus (VGE), and restenosis following stent placement, rotational atherectomy, and percutaneous transluminal coronary angioplasty (PTCA) (see, e.g., U.S. patent application publication no. 20070172483.) In one embodiment, a method of treating a human patient is provided, wherein the method comprises administering to the patient a stable aqueous solution based on the weight of the patient. For example, in one embodiment, a method of treating a human patient with PNH is provided, wherein the method comprises administering to the patient a stable aqueous solution (e.g., via intravenous infusion), wherein the stable aqueous solution has a pH of about 7.4 and comprises or consists of ravulizumab at a concentration of about 100 mg/mL, about 33.1 mM sodium phosphate, monobasic, about 16.5 mM sodium phosphate, dibasic, about 0.05% w/v polysorbate 80, about 25 mM L-arginine, about 50 mg/mL sucrose, and water for injection, wherein the stable aqueous solution is administered to the human patient:

(c) on Day 1 at a dose of:
2,400 mg to a patient weighing ≥40 kg to <60 kg,
2,700 mg to a patient weighing ≥60 kg to <100 kg, or
3,000 mg to a patient weighing ≥100 kg;
and

(b) on Day 15 and every eight weeks thereafter at a dose of:
3,000 mg to a patient weighing ≥40 kg to <60 kg,
3,300 mg to a patient weighing ≥60 kg to <100 kg, or
3,600 mg to a patient weighing ≥100 kg.
   In one embodiment, the stable aqueous solution is administered to a human patient weighing ≥40 kg to <60 kg on Day 1 at a dose of 2,400 mg and on Day 15 and every eight weeks thereafter at a dose of 3,000 mg. In another embodiment, the aqueous solution is administered to a human patient weighing ≥60 kg to <100 kg on Day 1 at a dose of 2,700 mg and on Day 15 and every eight weeks thereafter at a dose of 3,300 mg. In another embodiment, the stable aqueous solution is administered to a human patient weighing ≥100 kg on Day 1 at a dose of 3,000 mg and on Day 15 and every eight weeks thereafter at a dose of 3,600 mg.

In another embodiment, a method of treating a human patient with aH US is provided, wherein the method comprises administering to the patient a stable aqueous solution (e.g., via intravenous infusion), wherein the stable aqueous solution has a pH of about 7.4 and comprises or consists of ravulizumab at a concentration of about 100 mg/mL, about 33.1 mM sodium phosphate, monobasic, about 16.5 mM sodium phosphate, dibasic, about 0.05% w/v polysorbate 80, about 25 mM L-arginine, about 50 mg/mL sucrose, and water for injection, wherein the stable aqueous solution is administered to the human patient:

(d) on Day 1 at a dose of:
600 mg to a patient weighing ≥5 kg to <10 kg,
600 mg to a patient weighing ≥10 kg to <20 kg,
900 mg to a patient weighing ≥20 kg to <30 kg;
1,200 mg to a patient weighing ≥30 kg to <40 kg,
2,400 mg to a patient weighing ≥40 kg to <60 kg,
2,700 mg to a patient weighing ≥60 kg to <100 kg, or
3,000 mg to a patient weighing ≥100 kg; and

(b) on Day 15 and every four or eight weeks thereafter at a dose of:
300 mg to a patient weighing ≥5 kg to <10 kg,
600 mg to a patient weighing ≥10 kg to <20 kg,
2,100 mg to a patient weighing ≥20 kg to <30 kg;
2,700 mg to a patient weighing ≥30 kg to <40 kg,
3,000 mg to a patient weighing ≥40 kg to <60 kg,
3,300 mg to a patient weighing ≥60 kg to <100 kg, or
3,600 mg to a patient weighing ≥100 kg.
   In one embodiment, the stable aqueous solution is administered to a human patient weighing ≥5 kg to <10 kg on Day 1 at a dose of 600 mg and on Day 15 and every four weeks thereafter at a dose of 300 mg. In another embodiment, the stable aqueous solution is administered to a human patient weighing ≥10 kg to <20 kg on Day 1 at a dose of 600 mg and on Day 15 and every four weeks thereafter at a dose of 600 mg. In another embodiment, the stable aqueous solution is administered to a human patient weighing ≥20 kg to <30 kg on Day 1 at a dose of 900 mg and on Day 15 and every eight weeks thereafter at a dose of 2,100 mg. In another embodiment, the stable aqueous solution is administered to a human patient weighing ≥30 kg to <40 kg on Day 1 at a dose of 1,200 mg and on Day 15 and every eight weeks thereafter at a dose of 2,700 mg. In another embodiment, the stable aqueous solution is administered to a human patient weighing ≥40 kg to <60 kg on Day 1 at a dose of 2,400 mg and on Day 15 and every eight weeks thereafter at a dose of 3,000 mg. In another embodiment, the stable aqueous solution is administered to a human patient weighing ≥60 kg to <100 kg on Day 1 at a dose of 2,700 mg and on Day 15 and every eight weeks thereafter at a dose of 3,300 mg. In another embodiment, the stable aqueous solution is administered to a human patient weighing ≥100 kg on Day 1 at a dose of 3,000 mg and on Day 15 and every eight weeks thereafter at a dose of 3,600 mg. In another embodiment, the stable aqueous solution is supplied in a 3 mL single use vial comprising 300 mg of ravulizumab. In another embodiment, the stable aqueous solution is supplied in a 11 mL single use vial comprising 1,100 mg of ravulizumab.

In other embodiments, a method of treating a human patient is provided, wherein the method comprises administering to the patient a stable aqueous solution at a flat-fixed dose that is fixed irrespective of the weight of the patient. For example, the stable aqueous solution may be administered at a fixed dose of 1000, 1400, 1600, 1800, 2000, 2400, 3000, or 5400 mg, without regard to the patient's weight. In certain embodiments, dosage regimens are adjusted to provide the optimum desired response (e.g., an effective response).

In another embodiment, the stable aqueous solution is administered to a human patient at a dose of 3000 mg during the induction phase. In another embodiment, the stable aqueous solution is administered to a human patient one time at a dose of 3000 mg during the induction phase. In another embodiment, the stable aqueous solution is administered to a human patient at a dose of 5400 mg during the maintenance phase. In another embodiment, the stable aqueous solution is administered to a human patient three times at a dose of 5400 mg during the maintenance phase. For example, the anti-C5 antibody, or antigen binding fragment thereof, is administered at a dose of 5400 mg on Day 29 of the administration cycle and then every 84 days (or twelve weeks) thereafter during the maintenance phase, i.e., on days 113 and 197 of the administration cycle. In another embodiment, the stable aqueous solution is administered to a human patient at: (a) a dose of 3000 mg on Day 1, (b) a dose of 5400 mg four weeks thereafter, and then (c) at a dose of 5400 mg every 84 days (or twelve weeks) thereafter. In another embodiment, the stable aqueous solution is administered to a human patient at a dose of: (a) 3000 mg on Day 1 of the administration cycle during the induction phase and (b) 5400 mg on Day 29 of the administration cycle and then every 84 days (or twelve weeks) thereafter during the maintenance phase. In another embodiment, the stable aqueous solution is administered to a human patient at a dose of: (a) 3000 mg on Day 1 of the administration cycle during the induction phase and (b) 5400 mg on Days 29, 113, and 197 of the administration cycle during the maintenance phase. In another embodiment, the stable aqueous solution comprises ravulizumab at a concentration of 10 mg/mL. In another embodiment, the stable aqueous solution comprises ravulizumab at a concentration of 100 mg/mL

The infusion time and rate for administering the 100 mg/mL stable aqueous solution described herein is significantly reduced (e.g., by at least 50%) compared to the infusion time and rate for comparable lower dose (e.g., 10 mg/mL) solutions. The infusion time and rate can be adjusted by the clinician as deemed necessary. In one embodiment, the stable aqueous solution is administered to a human patient weighing ≥5 kg to <10 kg on Day 1 at a dose of 600 mg at a minimum infusion time of 1.4 hours and a maximum infusion rate of 8 mL/hour. In another embodiment, the stable aqueous solution is administered to a human patient weighing ≥5 kg to <10 kg on Day 15 and every four weeks thereafter at a dose of 300 mg at a minimum infusion time of 0.8 hours and a maximum infusion rate of 8 mL/hour. In another embodiment, the stable aqueous solution is administered to a human patient weighing ≥10 kg to <20 kg on Day 1 at a dose of 600 mg at a minimum infusion time of 0.8 hours and a maximum infusion rate of 16 mL/hour. In another embodiment, the stable aqueous solution is administered to a human patient weighing ≥10 kg to <20 kg on Day 15 and every four weeks thereafter at a dose of 600 mg at a minimum infusion time of 0.8 hours and a maximum infusion rate of 16 mL/hour. In another embodiment, the stable aqueous solution is administered to a human patient weighing ≥20 kg to <30 kg on Day 1 at a dose of 900 mg at a minimum infusion time of 0.6 hours and a maximum infusion rate of 30 mL/hour. In another embodiment, the stable aqueous solution is administered to a human patient weighing ≥20 kg to <30 kg on Day 15 and every eight weeks thereafter at a dose of 2,100 mg at a minimum infusion time of 1.3 hours and a maximum infusion rate of 33 mL/hour. In another embodiment, the stable aqueous solution is administered to a human patient weighing ≥30 kg to <40 kg on Day 1 at a dose of 1,200 mg at a minimum infusion time of 0.5 hours and a maximum infusion rate of 46 mL/hour. In another embodiment, the stable aqueous solution is administered to a human patient weighing ≥30 kg to <40 kg on Day 15 and every eight weeks thereafter at a dose of 2,700 mg at a minimum infusion time of 1.1 hours and a maximum infusion rate of 49 mL/hour. In another embodiment, the stable aqueous solution is administered to a human patient weighing ≥40 kg to <60 kg on Day 1 at a dose of 2,400 mg at a minimum infusion time of 0.8 hours and a maximum infusion rate of 64 mL/hour. In another embodiment, the stable aqueous solution is administered to a human patient weighing ≥40 kg to <60 kg on Day 15 and every eight weeks thereafter at a dose of 3,000 mg at a minimum infusion time of 0.9 hours and a maximum infusion rate of 65 mL/hour. In another embodiment, the stable aqueous solution is administered to a human patient weighing ≥60 kg to <100 kg on Day 1 at a dose of 2,700 mg at a minimum infusion time of 0.6 hours and a maximum infusion rate of 92 mL/hour. In another embodiment, the stable aqueous solution is administered to a human patient weighing ≥60 kg to <100 kg on Day 15 and every eight weeks thereafter at a dose of 3,300 mg at a minimum infusion time of 0.7 hours and a maximum infusion rate of 99 mL/hour. In another embodiment, the stable aqueous solution is administered to a human patient weighing ≥100 kg on Day 1 at a dose of 3,000 mg at a minimum infusion time of 0.4 hours and a maximum infusion rate of 144 mL/hour. In another embodiment, the stable aqueous solution is administered to a human patient weighing ≥100 kg on Day 15 and every eight weeks thereafter at a dose of 3,600 mg at a minimum infusion time of 0.5 hours and a maximum infusion rate of 144 mL/hour.

### IV. Combination Treatments

In one embodiment, the solutions described herein are administered to a patient as a monotherapy. In another embodiment, they are administered in conjunction with one or more additional agents and/or other therapies (e.g., which are suitable for treating complement-associated disorders). For example, the combination therapy can include administering to the human patient one or more additional agents (e.g., anti-coagulants, anti-hypertensives, or anti-inflammatory drugs (e.g., steroids)) that provide a therapeutic benefit to a patient. In one embodiment, the solutions described herein are administered in combination with an anti-inflammatory agent (e.g., NSAIDs, corticosteroids, methotrexate, hydroxychloroquine, anti-TNF agents such as etanercept and infliximab, a B cell depleting agent such as rituximab, an interleukin-1 antagonist, or a T cell costimulatory blocking agent such as abatacept).

Additional agents for treating a complement-associated disorder in a subject will vary depending on the particular disorder being treated, but can include, without limitation, one or more antihypertensives (e.g., an angiotensin-converting enzyme inhibitor, labetalol, hydralazine, nifedipine, calcium channel antagonists, nitroglycerin, or sodium nitroprussiate), anticoagulants, corticosteroids (e.g., prednisone), immunosuppressive agents (e.g., vincristine or cyclosporine A), anticoagulants (e.g., warfarin (Coumadin), aspirin, heparin, phenindione, fondaparinux, idraparinux), thrombin inhibitors (e.g., argatroban, lepirudin, bivalirudin, or dabigatran), fibrinolytic agents (e.g., ancrod, α-aminocaproic acid, antiplasmin-a₁, prostacyclin, and defibrotide), anti-hypertensive agents (e.g., labetalol, hydralazine, nifedipine, calcium channel antagonists, nitroglycerin, or sodium nitroprussiate), lipid-lowering agents (e.g., an inhibitor of hydroxymethylglutaryl CoA reductase), anti-seizure agents (e.g., magnesium sulfate), anti-thrombotic agents (e.g., heparin, antithrombin, prostacyclin, or low dose aspirin), sympathomimetics (e.g., albuterol), antibiotics, deoxyribonucleases (e.g., Pulmozyme®), anticholinergic drugs, anti-IgE inhibitors (e.g., anti-IgE antibodies), corticosteroids, or non-steroidal anti-inflammatory drugs (NSAID). Many different NSAIDS are available, some over the counter including ibuprofen (Advil®, Motrin®, Nuprin®) and naproxen (Alleve®) and many others are available by prescription including meloxicam (Mobic®), etodolac (Lodine®), nabumetone (Relafen®), sulindac (Clinoril®), tolementin (Tolectin®), choline magnesium salicylate (Trilasate®), diclofenac (Cataflam®, Voltaren®, Arthrotec®), Diflusinal (Dolobid®), indomethicin (Indocin®), ketoprofen (Orudis®, Oruvail®), oxaprozin (Daypro®), and piroxicam (Feldene®) (see, e.g., Mihu et al. (2007) J Gastrointestin Liver Dis 16(4):419-424). In another embodiment, a solution described herein can be formulated for administration to a patient along with intravenous gamma globulin therapy (IVIG), plasmapheresis, plasma replacement, or plasma exchange.

In one embodiment, the solution and one or more additional agents and/or therapies are administered at the same time. In another embodiment, the solution is administered prior to administration of one or more additional agents and/or therapies. In another embodiment, the solution is administered after administration of one or more additional agents and/or therapies. When an antibody solution described herein is used in combination with a second active agent, the agents (e.g., the anti-C5 antibody and second agent) can be formulated separately or together. For example, the solution and agent can be mixed, e.g., just prior to administration, and administered together or separately, e.g., at the same or different times.

### V. Kits and Unit Dosage Forms

Also provided herein are kits which include a stable aqueous solution containing an anti-C5 antibody, or antigen binding fragment thereof, such as ravulizumab, in a therapeutically effective amount, suitable for administration to a human patient (e.g., a patient having a complement-associated disorder). The kits optionally also can include instructions, e.g., comprising administration schedules, to allow a practitioner (e.g., a physician, nurse, or patient) to administer the composition contained therein to administer the solution to a patient.

The kits can also contain a suitable means for delivery of one or more solutions to a patient in need thereof, e.g., a patient afflicted with, suspected of having, or at risk for developing, a complement-associated disorder. In one embodiment, the means is suitable for intravenous delivery of the solution to the patient.

Optionally, the kits include multiple packages of a single-dose solution, each containing an effective amount of the solution for a single administration. Instruments or devices necessary for administering the solution also may be included in the kits. For instance, a kit may provide one or more pre-filled syringes containing the solution.

In one embodiment, the kit comprises: (i) any of the solutions described herein and (ii) instructions for use. In another embodiment, the kit comprises: (i) any of the solutions described herein, (ii) normal saline (0.9% sodium chloride); and (iii) instructions for use. In another embodiment, the kit comprises: (i) a formulation comprising or consisting of ravulizumab at a concentration of about 100 mg/mL; about 33.1 mM sodium phosphate, monobasic; about 16.5 mM sodium phosphate, dibasic; about 0.05% w/v polysorbate 80; about 25 mM L-arginine; and about 50 mg/mL sucrose; (ii) normal saline (0.9% sodium chloride); and (iii) instructions for use. In another embodiment, the formulation is lyophilized. In another embodiment, the kit includes a 3 mL single use vial comprising 300 mg of ravulizumab. In another embodiment, kit includes an 11 mL single use vial comprising 1,100 mg of ravulizumab.

### VI. Methods for Preparing the Highly-Concentrated Antibody Solutions

Also provided herein are methods for preparing a highly-concentrated anti-C5 antibody solution. In one embodiment, a method for producing a stable aqueous solution comprising ravulizumab at a concentration of no more than about 10 mg/mL; about 33.1 mM sodium phosphate, monobasic; about 16.5 mM sodium phosphate, dibasic; about 0.05% w/v polysorbate 80; about 25 mM L-arginine; about 50 mg/mL sucrose; and water for injection is provided, wherein the method comprises:

i) providing a first aqueous solution comprising about 10 mg/mL ravulizumab,

ii) subjecting the first aqueous solution to diafiltration into a formulation about 33.1 mM sodium phosphate, monobasic; about 16.5 mM sodium phosphate, dibasic; about 0.05% w/v polysorbate 80; about 25 mM L-arginine; about 50 mg/mL sucrose; and water for injection at pH 7.4 to thereby produce a second aqueous solution; and

iii) concentrating the second aqueous solution to produce a stable aqueous solution comprising ravulizumab at a concentration of about 100 mg/mL; about 33.1 mM sodium phosphate, monobasic; about 16.5 mM sodium phosphate, dibasic; about 0.05% w/v polysorbate 80; about 25 mM L-arginine; about 50 mg/mL sucrose; and water for injection.

Unless otherwise defined, all technical and scientific terms used herein have the same meaning as commonly understood by one of ordinary skill in the art to which the present disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries (e.g., PUBMED, NCBI or UNIPROT accession numbers), and other references mentioned herein are incorporated by reference in their entirety.

### EXAMPLES

### Example 1: Formulation

A sterile aqueous solution containing ULTOMIRIS® (ravulizumab) at a concentration of 100 mg/mL in 50 mM sodium phosphate, 25 mM L-arginine, 5% (w/v) sucrose, 0.05% (w/v) Polysorbate 80, at pH 7.4 was formulated in stoppered 3 ml and 11 mL glass vials. The solution is designed for infusion by diluting into commercially available normal saline (0.9% sodium chloride).

A. Drug Product

The quantitative and qualitative composition of the drug product are presented in Table 1. Excipients are tested to the United States Pharmacopeia (USP), European Pharmacopoeia (Ph. Eur.), and/or Japanese Pharmacopeia (JP). The ravulizumab vial content is based on the extractable volume.

TABLE 1
ULTOMIRIS ® (ravulizumab) Drug Product Composition

| Component | | | | |
|---|---|---|---|---|
| (Formulation | | | | |
| Concentration) | Quality Standard | Function | Amountvial | |
| Ravulizumab | In-House Standard | Active | 1100 mg | 300 mg |
| (100 mg/mL) | | Ingredient | | |
| Sodium phosphate | USP, Ph. Eur. | Buffering | 50.3 mg | 13.7 mg |
| monobasic | | Agent | | |
| 33.1 mM ₁ (4.57 mg/mL) | | | | |
| Sodium phosphate | USP, Ph. Eur. | Buffering | 48.6 mg | 13.3 mg |
| dibasic 16.5 mM ¹ | | Agent | | |
| (4.42 mg/mL) | | | | |
| L-arginine | USP, Ph. Eur., JP | Stabilizer | 47.6 mg | 13.0 mg |
| 25 mM (4.33 mg/mL) | | | | |
| Sucrose 50 mg/mL | NF, Ph. Eur., JP | Tonicifying | 550 mg | 150 mg |
| | | agent and | | |
| | | stabilizer | | |
| Polysorbate 80 | NF, Ph. Eur., JP | Surfactant | 5.5 mg | 1.5 mg |
| 0.05% w/v | | | | |
| Water for injection | USP, Ph. Eur., JP | Solvent | QS | QS |

¹ The sodium phosphate concentration is approximately 50 mm, which is comprised of sodium phosphate monobasic and the sodium phosphate dibasic.

The container closure system consists of a USP/Ph. Eur. Type 1 glass vial, a butyl rubber stopper laminated with Flurotec®, and an aluminum seal with a polypropylene flip-off cap.

ULTOMIRIS® (ravulizumab) injection 100 mg/mL is a sterile, translucent, clear to yellowish color, preservative-free solution for intravenous use. Each single-dose vial contains 300 mg or 1,100 mg ULTOMIRIS® (ravulizumab) at a concentration of 100 mg/mL with a pH of 7.4. Each mL also contains polysorbate 80 (0.5 mg) (vegetable origin), sodium chloride (8.77 mg), sodium phosphate dibasic (4.42 mg), sodium phosphate monobasic (4.57 mg), L-arginine (4.33 mg), sucrose (50 mg) and Water for Injection, USP.

The product is available as preservative-free solution supplied as single-use vial per carton at the following strengths:

TABLE 2
ULTOMIRIS ® (ravulizumab) Drug Product Composition

| | | Strength | National Drug |
|---|---|---|---|
| Concentration | Volume | per vial | Code (NDC) |
| 100 mg/mL | 3 mL | 300 mg | NDC 25682-025-01 |
| 100 mg/mL | 11 mL | 1,100 mg | NDC 25682-028-01 |

ULTOMIRIS® (ravulizumab) vials are stored refrigerated at 2° C.-8° C. (36° F.-46° F.) in the original carton to protect from light. They are not to be frozen or shaken.

B. Indications

ULTOMIRIS® (ravulizumab) is indicated for the treatment of adult patients with paroxysmal nocturnal hemoglobinuria (PNH) and the treatment of adults and pediatric patients one month of age and older with atypical hemolytic uremic syndrome (aHUS) to inhibit complement-mediated thrombotic microangiopathy (TMA). ULTOMIRIS® (ravulizumab) is not indicated for the treatment of patients with Shiga toxin E. coli related hemolytic uremic syndrome (STEC-HUS).

C. Dosage and Administration

Patients are vaccinated for meningococcal disease according to current ACIP guidelines to reduce the risk of serious infection. Two weeks of antibacterial drug prophylaxis is provided to patients if ULTOMIRIS® (ravulizumab) is initiated immediately and vaccines are administered less than 2 weeks before starting ULTOMIRIS® (ravulizumab) therapy.

The recommended dosing regimen in adult patients with PNH weighing 40 kg or greater, consists of a loading dose followed by maintenance dosing, administered by intravenous infusion. The doses are administered based on the patient's body weight, as shown in Table 3. Starting 2 weeks after the loading dose administration, maintenance doses are started at a once every 8-week interval. The dosing schedule can occasionally vary within 7 days of the scheduled infusion day (except for the first maintenance dose of ULTOMIRIS® (ravulizumab); but the subsequent doses should be administered according to the original schedule.

TABLE 3
ULTOMIRIS ® (ravulizumab) Weight-Based Dosing Regimen-PNH

| | Loading | Maintenance Dose (mg) | |
|---|---|---|---|
| Body Weight Range (kg) | Dose (mg) | and Dosing Interval | |
| greater than or equal to 40 | 2,400 | 3,000 | Every 8 weeks |
| to less than 60 | | | |
| greater than or equal to 60 | 2,700 | 3,300 | |
| to less than 100 | | | |
| greater than or equal to 100 | 3,000 | 3,600 | |

The recommended dosing regimen in adult and pediatric patients one month of age and older with aHUS weighing 5 kg or greater, consists of a loading dose, followed by maintenance dosing, administered by intravenous infusion. The doses are administered based on the patient's body weight, as shown in Table 4. Starting 2 weeks after the loading dose administration, maintenance doses begin once every 8 weeks or every 4 weeks (depending on body weight). The dosing schedule can occasionally vary within 7 days of the scheduled infusion day (except for the first maintenance dose of ULTOMIRIS® (ravulizumab)); but the subsequent doses are administered according to the original schedule.

TABLE 4
ULTOMIRIS ® (ravulizumab) Weight-Based Dosing Regimen-aHUS

| | Loading | Maintenance Dose (mg) | |
|---|---|---|---|
| Body Weight Range (kg) | Dose (mg) | and Dosing Interval | |
| greater than or equal to 5 to | 600 | 300 | Every 4 weeks |
| less than 10 | | | |
| greater than or equal to 10 | 600 | 600 | |
| to less than 20 | | | |
| greater than or equal to 20 | 900 | 2,100 | Every 8 weeks |
| to less than 30 | | | |
| greater than or equal to 30 | 1,200 | 2,700 | |
| to less than 40 | | | |
| greater than or equal to 40 | 2,400 | 3,000 | |
| to less than 60 | | | |
| greater than or equal to 60 | 2,700 | 3,300 | |
| to less than 100 | | | |
| greater than or equal to 100 | 3,000 | 3,600 | |

For patients switching from SOLIRIS® (eculizumab) to ULTOMIRIS® (ravulizumab), the loading dose of ULTOMIRIS® (ravulizumab) is administered 2 weeks after the last eculizumab infusion, and then maintenance doses are administered once every 8 weeks or every 4 weeks (depending on body weight), starting 2 weeks after loading dose administration. Administration of PE/PI (plasmapheresis or plasma exchange, or fresh frozen plasma infusion) can reduce ULTOMIRIS® (ravulizumab) serum levels.

D. Preparation

Each vial of ULTOMIRIS® (ravulizumab) is intended for single-dose only. ULTOMIRIS® (ravulizumab) is supplied in 300 mg/3 mL (100 mg/mL) in a single-dose vial and 1,100 mg/11 mL (100 mg/mL) in a single-dose vial. ULTOMIRIS® (ravulizumab) 100 mg/mL (3 mL and 11 mL vials) and 10 mg/mL (30 mL vials) should not be mixed together.

ULTOMIRIS® (ravulizumab) requires dilution to a final concentration of 50 mg/mL for the 3 mL and 11 mL vials or a final concentration of 5 mg/mL for the 30 mL vials. Aseptic technique is used to prepare ULTOMIRIS as follows:
1. The number of vials diluted is determined based on the individual patient's weight and the prescribed dose.
2. Prior to dilution, the solution is visually inspected in the vials; the solution should be free of any particulate matter or precipitation. The solution is not used if there is evidence of particulate matter or precipitation.
3. The calculated volume of ULTOMIRIS® (ravulizumab) is withdrawn from the appropriate number of vials and diluted in an infusion bag using 0.9% Sodium Chloride Injection, USP to a final concentration of 50 mg/mL for the for the 3 mL and 11 mL vials or a final concentration of 5 mg/mL for the 30 mL vials. The product is mixed gently. It should notbe frozen or shaken and should be protected from light.
4. The prepared solution is administered immediately following preparation. Infusion is administered through a 0.2 or 0.22 micron filter.
5. If the diluted ULTOMIRIS® (ravulizumab) infusion solution is not used immediately, it is stored in refrigeration at 2° C.-8° C. (36° F.-46° F.) and must not exceed 24 hours, taking into account the expected infusion time. Once removed from refrigeration, the diluted ULTOMIRIS® (ravulizumab) infusion solution is administered within 6 hours if prepared with ULTOMIRIS® (ravulizumab) 3 mL or 11 mL vials.

E. Administration

ULTOMIRIS® (ravulizumab) is administered as an intravenous infusion. ULTOMIRIS® (ravulizumab) is diluted to a final concentration of 50 mg/mL for the 100 mg/mL formulation (3 mL and 11 mL vials). ULTOMIRIS® (ravulizumab) is administered through a 0.2 or 0.22 micron filter. The loading dose and maintenance doses for ULTOMIRIS® (ravulizumab) 100 mg/ml (3 mL and 11 mL vials) are set forth below in Tables 5 and 6.

TABLE 5
Loading Dose Administration Reference Table for ULTOMIRIS ®
(ravulizumab) 100 mg/mL (3 mL and 11 mL vials)

| | | | Volume | | Min. | Max. |
|---|---|---|---|---|---|---|
| | | | of NaCl | Total | Infusion | Infusion |
| Body Weight | Loading | ULTOMIRIS | Diluent^{b} | Volume | Time | Rate |
| Range (kg)^{a} | Dose (mg) | Volume (mL) | (mL) | (mL) | (hr) | (mL/hr) |
| greater than or equal | 600 | 6 | 6 | 12 | 1.4 | 8 |
| to 5 to less than 10 | | | | | | |
| greater than or equal | 600 | 6 | 6 | 12 | 0.8 | 16 |
| to 10 to less than 20 | | | | | | |
| greater than or equal | 900 | 9 | 9 | 18 | 0.6 | 30 |
| to 20 to less than 30 | | | | | | |
| greater than or equal | 1,200 | 12 | 12 | 24 | 0.5 | 46 |
| to 30 to less than 40 | | | | | | |
| greater than or equal | 2,400 | 24 | 24 | 48 | 0.8 | 64 |
| to 40 to less than 60 | | | | | | |
| greater than or equal | 2,700 | 27 | 27 | 54 | 0.6 | 92 |
| to 60 to less than 100 | | | | | | |
| greater than or | 3,000 | 30 | 30 | 60 | 0.4 | 144 |
| equal to 100 | | | | | | |

^{a}Body weight at time of treatment
^{b}Dilute ULTOMIRIS only using 0.9% Sodium Chloride Injection, USP.

TABLE 6
Maintenance Dose Administration Reference Table for ULTOMIRIS ®
(ravulizumab) 100 mg/mL (3 mL and 11 mL vials)

| | | | Volume | | Minimum | Maximum |
|---|---|---|---|---|---|---|
| | | | of NaCl | Total | Infusion | Infusion |
| Body Weight | Maint. | ULTOMIRIS | Diluent^{b} | Volume | Time | Rate |
| Range (kg)^{a} | Dose (mg) | Volume (mL) | (mL) | (mL) | (hr) | (mL/hr) |
| greater than or equal | 300 | 3 | 3 | 6 | 0.8 | 8 |
| to 5 to less than 10 | | | | | | |
| greater than or equal | 600 | 6 | 6 | 12 | 0.8 | 16 |
| to 10 to less than 20 | | | | | | |
| greater than or equal | 2,100 | 21 | 21 | 42 | 1.3 | 33 |
| to 20 to less than 30 | | | | | | |
| greater than or equal | 2,700 | 27 | 27 | 54 | 1.1 | 49 |
| to 30 to less than 40 | | | | | | |
| greater than or equal | 3,000 | 30 | 30 | 60 | 0.9 | 65 |
| to 40 to less than 60 | | | | | | |
| greater than or equal | 3,300 | 33 | 33 | 66 | 0.7 | 99 |
| to 60 to less than 100 | | | | | | |
| greater than or | 3,600 | 36 | 36 | 72 | 0.5 | 144 |
| equal to 100 | | | | | | |

^{a}Body weight at time of treatment
^{b}Dilute ULTOMIRIS only using 0.9% Sodium Chloride Injection, USP.

Prior to administration, the admixture is allowed to adjust to room temperature (18° C.-25° C., 64° F.-77° F.). The admixture is not heated with any heat source other than ambient air temperature.

If an adverse reaction occurs during the administration of ULTOMIRIS® (ravulizumab), the infusion can be slowed or stopped at the discretion of the physician. The patient is monitored for at least one hour following completion of the infusion for signs or symptoms of an infusion reaction.

### Example 2: Interim Analysis of Phase 2 Study Evaluating Efficacy, Safety, and Pharmacokinetics of Intravenous Ravulizumab 100 mg/ml Formulation in Patients with PNH

ULTOMIRIS® (ravulizumab) demonstrated proven efficacy (e.g., intravascular hemolysis control, transfusion independence, and minimized breakthrough hemolysis) and safety in two large phase 3 trials and real-world PNH populations. Accordingly, an interim analysis of a phase 2, open-label, multiple ascending dose study extension period (NCT02605993) was conducted to characterize the efficacy, safety, pharmacokinetics (PK) and immunogenicity of ULTOMIRIS® (ravulizumab) following a formulation change (10 mg/mL intravenous [IV] to 100 mg/mL IV).

In brief, complement inhibitor-naive adults with PNH received four different ULTOMIRIS® (ravulizumab) dosing regimens (cohorts 1-4; see FIGURE) according to the protocol described in WO 2017/123636, the contents of which are expressly incorporated herein by reference. In the extension phase, cohorts 1-3 switched to weight-based dosing, followed by all cohorts changing from ULTOMIRIS® (ravulizumab) 10 mg/mL IV to 100 mg/mL IV later in the extension period. Data before and after changing the formulation were then compared. Efficacy (lactate dehydrogenase levels), safety, PK (serum ULTOMIRIS® (ravulizumab) trough concentrations) and immunogenicity were assessed. Analyses for cohorts 1-3 (received weight-based dosing every 8 weeks [q8w]) were grouped. Results for cohort 4 (dosed every 12 weeks) were presented separately. Adverse events (AEs) were coded using Medical Dictionary for Regulatory Activities version 21.0. Patients gave informed consent, and the relevant independent ethics committees provided approval.

In total, 25 patients receiving ULTOMIRIS® (ravulizumab) 10 mg/mL IV switched to 100 mg/mL IV. The majority of patients were male (76.0%, n=19). Ethnicity was reported as not Hispanic or Latino (84.0%, n=21), Hispanic or Latino (4.0%, n=1) or was not provided (12.0%, n=3). Patients reported their race as White (60.0%, n=15), Asian (24.0%, n=6), ‘Other’ (4.0%, n=1) or did not disclose this information (12.0%, n=3). Mean (standard deviation [SD]) age at first infusion was 44.6 (16.5) years. At baseline, mean (SD) weight, height and body mass index were 78.1 (14.8) kg, 174.8 (10.6) cm and 25.4 (3.2) kg/m², respectively. No meaningful difference in mean lactate dehydrogenase levels or serum trough concentrations following formulation change was observed. Overall exposure-adjusted rate of treatment emergent AEs (TEAEs) and serious AEs were comparable for both formulations, and all reported TEAEs were considered unrelated to ULTOMIRIS® (ravulizumab). No deaths, discontinuations or anti-drug antibody positive responses occurred before or after formulation change. Use of the 100 mg/mL formulation gave an 87-minute reduction in infusion time versus the 10 mg/mL preparation for adult patients weighing 40-60 kg (57 minutes [100 mg/mL] vs 144 minutes [10 mg/mL]). Infusion time was reduced by 78 minutes for patients weighing 60-100 kg (42 minutes [100 mg/mL] vs 120 minutes [10 mg/mL]), and by 102 minutes for patients weighing >100 kg (30 minutes [100 mg/mL] vs 132 minutes [10 mg/mL]).

In this interim analysis, the efficacy, safety, PK and immunogenicity of ULTOMIRIS® (ravulizumab) IV 100 mg/mL were comparable to the IV 10 mg/mL formulation, and the high-concentration formulation was well tolerated in patients with PNH. The ULTOMIRIS® (ravulizumab) IV 100 mg/mL formulation permitted a considerable reduction in infusion time versus the low-concentration formulation (78-102 minute reduction of maintenance dose infusion time in adults versus 10 mg/mL formulation), offering improved patient convenience.

SEQUENCE SUMMARY

SEQ ID NO: 1
GYIFSNYWIQ
SEQ ID NO: 2
EILPGSGSTEYTENFKD
SEQ ID NO: 3
YFFGSSPNWYFDV
SEQ ID NO: 4
GASENIYGALN
SEQ ID NO: 5
GATNLAD
SEQ ID NO: 6
QNVLNTPLT
SEQ ID NO: 7
QVQLVQSGAEVKKPGASVKVSCKASGYIFSNYWIQWVRQAPGQGLEWM
GEILPGSGSTEYTENFKDRVTMTRDTSTSTVYMELSSLRSEDTAVYYCA
RYFFGSSPNWYFDVWGQGTLVTVSS
SEQ ID NO: 8
DIQMTQSPSSLSASVGDRVTITCGASENIYGALNWYQQKPGKAPKLLIY
GATNLADGVPSRFSGSGSGTDFTLTISSLQPEDFATYYCQNVLNTPLTF
GQGTKVEIK
SEQ ID NO: 9
ASTKGPSVFPLAPCSRSTSESTAALGCLVKDYFPEPVTVSWNSGALTSG
VHTFPAVLQSSGLYSLSSVVTVPSSNFGTQTYTCNVDHKPSNTKVDKTV
ERKCCVECPPCPAPPVAGPSVFLFPPKPKDTLMISRTPEVTCVVVDVSQ
EDPEVQFNWYVDGVEVHNAKTKPREEQFNSTYRVVSVLTVLHQDWLNGK
EYKCKVSNKGLPSSIEKTISKAKGQPREPQVYTLPPSQEEMTKNQVSLT
CLVKGFYPSDIAVEWESNGQPENNYKTTPPVLDSDGSFFLYSRLTVDKS
RWQEGNVFSCSVMHEALHNHYTQKSLSLSLGK
SEQ ID NO: 10
QVQLVQSGAEVKKPGASVKVSCKASGYIFSNYWIQWVRQAPGQGLEWM
GEILPGSGSTEYTENFKDRVTMTRDTSTSTVYMELSSLRSEDTAVYYCA
RYFFGSSPNWYFDVWGQGTLVTVSSASTKGPSVFPLAPCSRSTSESTAA
LGCLVKDYFPEPVTVSWNSGALTSGVHTFPAVLQSSGLYSLSSVVTVPS
SNFGTQTYTCNVDHKPSNTKVDKTVERKCCVECPPCPAPPVAGPSVFLF
PPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQK
SLSLSLGK
SEQ ID NO: 11
DIQMTQSPSSLSASVGDRVTITCGASENIYGALNWYQQKPGKAPKLLIY
GATNLADGVPSRFSGSGSGTDFTLTISSLQPEDFATYYCQNVLNTPLTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
SEQ ID NO: 12
QVQLVQSGAEVKKPGASVKVSCKASGHIFSNYWIQWVRQAPGQGLEW
MGEILPGSGHTEYTENFKDRVTMTRDTSTSTVYMELSSLRSEDTAVYYC
ARYFFGSSPNWYFDVWGQGTLVTVSS
SEQ ID NO: 13
ASTKGPSVFPLAPCSRSTSESTAALGCLVKDYFPEPVTVSWNSGALTSG
VHTFPAVLQSSGLYSLSSVVTVPSSNFGTQTYTCNVDHKPSNTKVDKTV
ERKCCVECPPCPAPPVAGPSVFLFPPKPKDTLMISRTPEVTCVVVDVSQ
EDPEVQFNWYVDGVEVHNAKTKPREEQFNSTYRVVSVLTVLHQDWLNGK
EYKCKVSNKGLPSSIEKTISKAKGQPREPQVYTLPPSQEEMTKNQVSLT
CLVKGFYPSDIAVEWESNGQPENNYKTTPPVLDSDGSFFLYSRLTVDKS
RWQEGNVFSCSVLHEALHSHYTQKSLSLSLGK
SEQ ID NO: 14
QVQLVQSGAEVKKPGASVKVSCKASGHIFSNYWIQWVRQAPGQGLEWM
GEILPGSGHTEYTENFKDRVTMTRDTSTSTVYMELSSLRSEDTAVYYCA
RYFFGSSPNWYFDVWGQGTLVTVSSASTKGPSVFPLAPCSRSTSESTAA
LGCLVKDYFPEPVTVSWNSGALTSGVHTFPAVLQSSGLYSLSSVVTVPS
SNFGTQTYTCNVDHKPSNTKVDKTVERKCCVECPPCPAPPVAGPSVFLF
PPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENN
YKTTPPVLDSDGSFFLYSRLTVDKSRWQEGNVFSCSVLHEALHSHYTQK
SLSLSLGK
SEQ ID NO: 15
ASTKGPSVFPLAPCSRSTSESTAALGCLVKDYFPEPVTVSWNSGALTSG
VHTFPAVLQSSGLYSLSSVVTVTSSNFGTQTYTCNVDHKPSNTKVDKTV
ERKCCVECPPCPAPPVAGPSVFLFPPKPKDTLYITREPEVTCVVVDVSH
EDPEVQFNWYVDGMEVHNAKTKPREEQFNSTFRVVSVLTVVHQDWLNGK
EYKCKVSNKGLPAPIEKTISKTKGQPREPQVYTLPPSREEMTKNQVSLT
CLVKGFYPSDIAVEWESNGQPENNYKTTPPMLDSDGSFFLYSKLTVDKS
RWQQGNVFSCSVMHEALHNHYTQKSLSLSPGK
SEQ ID NO: 16
QVQLVQSGAEVKKPGASVKVSCKASGYIFSNYWIQWVRQAPGQGLEWM
GEILPGSGSTEYTENFKDRVTMTRDTSTSTVYMELSSLRSEDTAVYYCA
RYFFGSSPNWYFDVWGQGTLVTVSSASTKGPSVFPLAPCSRSTSESTAA
LGCLVKDYFPEPVTVSWNSGALTSGVHTFPAVLQSSGLYSLSSVVTVTS
SNFGTQTYTCNVDHKPSNTKVDKTVERKCCVECPPCPAPPVAGPSVFLF
PPKPKDTLYITREPEVTCVVVDVSHEDPEVQFNWYVDGMEVHNAKTKPR
EEQFNSTFRVVSVLTVVHQDWLNGKEYKCKVSNKGLPAPIEKTISKTKG
QPREPQVYTLPPSREEMTKNQVSLTCLVKGFYPSDIAVEWESNGQPENN
YKTTPPMLDSDGSFFLYSKLTVDKSRWQQGNVFSCSVMHEALHNHYTQK
SLSLSPGK
SEQ ID NO: 17
GASENIYHALN
SEQ ID NO: 18
EILPGSGHTEYTENFKD
SEQ ID NO: 19
GHIFSNYWIQ
SEQ ID NO: 20
QVQLVQSGAEVKKPGASVKVSCKASGHIFSNYWIQWVRQAPGQGLEW
MGEILPGSGHTEYTENFKDRVTMTRDTSTSTVYMELSSLRSEDTAVYYC
ARYFFGSSPNWYFDVWGQGTLVTVSSASTKGPSVFPLAPCSRSTSESTA
ALGCLVKDYFPEPVTVSWNSGALTSGVHTFPAVLQSSGLYSLSSVVTVP
SSNFGTQTYTCNVDHKPSNTKVDKTVERKCCVECPPCPAPPVAGPSVFL
FPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAK
GQPREPQVYTLPPSQEEMTKNQVSLTCLVKGFYPSDIAVEWESNGQPEN
NYKTTPPVLDSDGSFFLYSRLTVDKSRWQEGNVFSCSVMHEALHNHYTQ
KSLSLSLGK

1. A stable aqueous solution comprising:
(a) ravulizumab at a concentration of about 100 mg/mL;
(b) about 50 mM sodium phosphate;
(c) about 0.05% w/v polysorbate 80;
(d) about 25 mM L-arginine;
(e) about 50 mg/mL sucrose; and
(f) water for injection;
wherein the solution has a pH of about 7.4 and is contained in a 3 mL single use vial comprising 300 mg of ravulizumab.

2. A stable aqueous solution consisting of:
(a) ravulizumab at a concentration of about 100 mg/mL;
(b) about 50 mM sodium phosphate;
(c) about 0.05% w/v polysorbate 80;
(d) about 25 mM L-arginine;
(e) about 50 mg/mL sucrose; and
(f) water for injection;
wherein the solution has a pH of about 7.4 and is contained in a 3 mL single use vial comprising 300 mg of ravulizumab.

3. A stable aqueous solution comprising:
(a) ravulizumab at a concentration of about 100 mg/mL;
(b) about 33.1 mM sodium phosphate, monobasic;
(c) about 16.5 mM sodium phosphate, dibasic;
(d) about 0.05% w/v polysorbate 80;
(f) about 25 mM L-arginine;
(g) about 50 mg/mL sucrose; and
(h) water for injection;
wherein the solution has a pH of about 7.4 and is contained in a 3 mL single use vial comprising 300 mg of ravulizumab.

4. A stable aqueous solution consisting of:
(a) ravulizumab at a concentration of about 100 mg/mL;
(b) about 33.1 mM sodium phosphate, monobasic;
(c) about 16.5 mM sodium phosphate, dibasic;
(d) about 0.05% w/v polysorbate 80;
(f) about 25 mM L-arginine;
(g) about 50 mg/mL sucrose; and
(h) water for injection;
wherein the solution has a pH of about 7.4 and is contained in a 3 mL single use vial comprising 300 mg of ravulizumab.

5. A stable aqueous solution consisting of:
(a) ravulizumab at a concentration of 100 mg/mL;
(b) 33.1 mM sodium phosphate, monobasic;
(c) 16.5 mM sodium phosphate, dibasic;
(d) 0.05% w/v polysorbate 80;
(f) 25 mM L-arginine;
(g) 50 mg/mL sucrose; and
(h) water for injection;
wherein the solution has a pH of 7.4 and is contained in a 3 mL single use vial comprising 300 mg of ravulizumab.

6. A stable aqueous solution comprising:
(a) ravulizumab at a concentration of about 100 mg/mL;
(b) about 50 mM sodium phosphate;
(c) about 0.05% w/v polysorbate 80;
(d) about 25 mM L-arginine;
(e) about 50 mg/mL sucrose; and
(f) water for injection;
wherein the solution has a pH of about 7.4 and is contained in a 11 mL single use vial comprising 1100 mg of ravulizumab.

7. A stable aqueous solution consisting of:
(a) ravulizumab at a concentration of about 100 mg/mL;
(b) about 50 mM sodium phosphate;
(c) about 0.05% w/v polysorbate 80;
(d) about 25 mM L-arginine;
(e) about 50 mg/mL sucrose; and
(f) water for injection;
wherein the solution has a pH of about 7.4 and is contained in a 11 mL single use vial comprising 1100 mg of ravulizumab.

8. A stable aqueous solution comprising:
(a) ravulizumab at a concentration of about 100 mg/mL;
(b) about 33.1 mM sodium phosphate, monobasic;
(c) about 16.5 mM sodium phosphate, dibasic;
(d) about 0.05% w/v polysorbate 80;
(f) about 25 mM L-arginine;
(g) about 50 mg/mL sucrose; and
(h) water for injection;
wherein the solution has a pH of about 7.4 and is contained in a 11 mL single use vial comprising 1100 mg of ravulizumab.

9. A stable aqueous solution consisting of:
(a) ravulizumab at a concentration of about 100 mg/mL;
(b) about 33.1 mM sodium phosphate, monobasic;
(c) about 16.5 mM sodium phosphate, dibasic;
(d) about 0.05% w/v polysorbate 80;
(f) about 25 mM L-arginine;
(g) about 50 mg/mL sucrose; and
(h) water for injection;
wherein the solution has a pH of about 7.4 and is contained in a 11 mL single use vial comprising 1100 mg of ravulizumab.

10. A stable aqueous solution consisting of:
(a) ravulizumab at a concentration of 100 mg/mL;
(b) 33.1 mM sodium phosphate, monobasic;
(c) 16.5 mM sodium phosphate, dibasic;
(d) 0.05% w/v polysorbate 80;
(f) 25 mM L-arginine;
(g) 50 mg/mL sucrose; and
(h) water for injection;
wherein the solution has a pH of 7.4 and is contained in a 11 mL single use vial comprising 1100 mg of ravulizumab.

11. The stable aqueous solution of any one of the preceding claims, wherein the solution is sterile.

12. The stable aqueous solution of any one of the preceding claims, wherein the solution is a sterile, translucent, clear to yellowish color, preservative-free solution.

13. The stable aqueous solution of any one of the preceding claims, wherein the solution has a shelf-life of 18 months.

14. The stable aqueous solution of any of the preceding claims, wherein ravulizumab remains at least 97% monomeric during storage at 2° C. to 8° C. for at least six months as determined by SEC-HPLC.

15. The stable aqueous solution of any one of the preceding claims, wherein less than 3% of ravulizumab in the solution is aggregated as determined by SEC-HPLC.

16. The stable aqueous solution of any one of the preceding claims, wherein the solution is diluted prior to administration to a concentration of 50 mg/mL ravulizumab.

17. The stable aqueous solution of claim 16, wherein the solution is diluted prior to administration with normal saline (0.9% sodium chloride).

18. The stable aqueous solution of claim 16 or 17, wherein the solution is stable after dilution for up to 24 hours at 2° C.-8° C. (36° F.-46° F.).

19. The stable aqueous solution of any one of claims 1-18, wherein the solution is suitable for administration by intravenous infusion.

20. A method of treating a human patient with a complement-associated condition, comprising administering to the patient the stable aqueous solution of any one of claims 1-19 in an amount effective to treat the complement-associated condition.

21. The method of claim 20, wherein the complement-associated condition is selected from the group consisting of rheumatoid arthritis, antiphospholipid antibody syndrome, lupus nephritis, ischemia-reperfusion injury, thrombotic microangiopathy (TMA), atypical hemolytic uremic syndrome (aHUS), typical hemolytic uremic syndrome, paroxysmal nocturnal hemoglobinuria (PNH), dense deposit disease, neuromyelitis optica, multifocal motor neuropathy, multiple sclerosis, macular degeneration, HELLP syndrome, spontaneous fetal loss, thrombotic thrombocytopenic purpura, Pauci-immune vasculitis, epidermolysis bullosa, recurrent fetal loss, traumatic brain injury, myocarditis, a cerebrovascular disorder, a peripheral vascular disorder, a renovascular disorder, a mesenteric/enteric vascular disorder, vasculitis, Henoch-Schönlein purpura nephritis, systemic lupus erythematosus-associated vasculitis, vasculitis associated with rheumatoid arthritis, immune complex vasculitis, Takayasu's disease, dilated cardiomyopathy, diabetic angiopathy, Kawasaki's disease, venous gas embolus, restenosis following stent placement, rotational atherectomy, percutaneous transluminal coronary angioplasty, myasthenia gravis, cold agglutinin disease, dermatomyositis, paroxysmal cold hemoglobinuria, antiphospholipid syndrome, Graves' disease, atherosclerosis, Alzheimer's disease, systemic inflammatory response sepsis, septic shock, spinal cord injury, glomerulonephritis, transplant rejection, Hashimoto's thyroiditis, type I diabetes, psoriasis, pemphigus, autoimmune hemolytic anemia, idiopathic thrombocytopenic purpura, Goodpasture's syndrome, Degos disease, and catastrophic antiphospholipid syndrome.

22. The method of claim 21, wherein the complement-associated condition is TMA.

23. The method of claim 21, wherein the complement-associated condition is aHUS.

24. The method of claim 21, wherein the complement-associated condition is PNH.

25. A method of treating a human patient with PNH, comprising administering to the patient a stable aqueous solution, wherein the stable aqueous solution has a pH of about 7.4 and comprises or consists of ravulizumab at a concentration of about 100 mg/mL, about 33.1 mM sodium phosphate, monobasic, about 16.5 mM sodium phosphate, dibasic, about 0.05% w/v polysorbate 80, about 25 mM L-arginine, about 50 mg/mL sucrose, and water for injection, wherein the stable aqueous solution is administered to the human patient:
(e) on Day 1 at a dose of:
2,400 mg to a patient weighing ≥40 kg to <60 kg,
2,700 mg to a patient weighing ≥60 kg to <100 kg, or
3,000 mg to a patient weighing ≥100 kg;
and
(b) on Day 15 and every eight weeks thereafter at a dose of:
3,000 mg to a patient weighing ≥40 kg to <60 kg,
3,300 mg to a patient weighing ≥60 kg to <100 kg, or
3,600 mg to a patient weighing ≥100 kg.

26. The method of claim 25, wherein the stable aqueous solution is administered to a human patient weighing ≥40 kg to <60 kg on Day 1 at a dose of 2,400 mg and on Day 15 and every eight weeks thereafter at a dose of 3,000 mg.

27. The method of claim 25, wherein the stable aqueous solution is administered to a human patient weighing ≥60 kg to <100 kg on Day 1 at a dose of 2,700 mg and on Day 15 and every eight weeks thereafter at a dose of 3,300 mg.

28. The method of claim 25, wherein the stable aqueous solution is administered to a human patient weighing ≥100 kg on Day 1 at a dose of 3,000 mg and on Day 15 and every eight weeks thereafter at a dose of 3,600 mg.

29. A method of treating a human patient with aHUS, comprising administering to the patient a stable aqueous solution, wherein the stable aqueous solution has a pH of about 7.4 and comprises or consists of ravulizumab at a concentration of about 100 mg/mL, about 33.1 mM sodium phosphate, monobasic, about 16.5 mM sodium phosphate, dibasic, about 0.05% w/v polysorbate 80, about 25 mM L-arginine, about 50 mg/mL sucrose, and water for injection, wherein the stable aqueous solution is administered to the human patient:
(f) on Day 1 at a dose of:
600 mg to a patient weighing ≥5 kg to <10 kg,
600 mg to a patient weighing ≥10 kg to <20 kg,
900 mg to a patient weighing ≥20 kg to <30 kg;
1,200 mg to a patient weighing ≥30 kg to <40 kg,
2,400 mg to a patient weighing ≥40 kg to <60 kg,
2,700 mg to a patient weighing ≥60 kg to <100 kg, or
3,000 mg to a patient weighing ≥100 kg; and
(b) on Day 15 and every four or eight weeks thereafter at a dose of:
300 mg to a patient weighing ≥5 kg to <10 kg,
600 mg to a patient weighing ≥10 kg to <20 kg,
2,100 mg to a patient weighing ≥20 kg to <30 kg;
2,700 mg to a patient weighing ≥30 kg to <40 kg,
3,000 mg to a patient weighing ≥40 kg to <60 kg,
3,300 mg to a patient weighing ≥60 kg to <100 kg, or
3,600 mg to a patient weighing ≥100 kg.

30. The method of claim 29, wherein the stable aqueous solution is administered to a human patient weighing ≥5 kg to <10 kg on Day 1 at a dose of 600 mg and on Day 15 and every four weeks thereafter at a dose of 300 mg.

31. The method of claim 29, wherein the stable aqueous solution is administered to a human patient weighing ≥10 kg to <20 kg on Day 1 at a dose of 600 mg and on Day 15 and every four weeks thereafter at a dose of 600 mg.

32. The method of claim 29, wherein the stable aqueous solution is administered to a human patient weighing ≥20 kg to <30 kg on Day 1 at a dose of 900 mg and on Day 15 and every eight weeks thereafter at a dose of 2,100 mg.

33. The method of claim 29, wherein the stable aqueous solution is administered to a human patient weighing ≥30 kg to <40 kg on Day 1 at a dose of 1,200 mg and on Day 15 and every eight weeks thereafter at a dose of 2,700 mg.

34. The method of claim 29, wherein the stable aqueous solution is administered to a human patient weighing ≥40 kg to <60 kg on Day 1 at a dose of 2,400 mg and on Day 15 and every eight weeks thereafter at a dose of 3,000 mg.

35. The method of claim 29, wherein the stable aqueous solution is administered to a human patient weighing ≥60 kg to <100 kg on Day 1 at a dose of 2,700 mg and on Day 15 and every eight weeks thereafter at a dose of 3,300 mg.

36. The method of claim 29, wherein the stable aqueous solution is administered to a human patient weighing ≥100 kg on Day 1 at a dose of 3,000 mg and on Day 15 and every eight weeks thereafter at a dose of 3,600 mg.

37. The method of any one of claims 20-36, wherein stable aqueous solution is supplied in a 3 mL single use vial comprising 300 mg of ravulizumab.

38. The method of any one of claims 20-36, wherein stable aqueous solution is supplied in a 11 mL single use vial comprising 1,100 mg of ravulizumab.

39. The method of any one of claims 20-36, wherein the stable aqueous solution is administered by intravenous infusion.

40. A therapeutic kit comprising:
(i) the stable aqueous solution according to any one of claims 1-19; and
(ii) instructions for use.

41. A therapeutic kit comprising:
(i) the stable aqueous solution according to any one of claims 1-19;
(ii) normal saline (0.9% sodium chloride); and
(iii) instructions for use.

42. A therapeutic kit comprising:
(i) a formulation comprising or consisting of ravulizumab at a concentration of about 100 mg/mL; about 33.1 mM sodium phosphate, monobasic; about 16.5 mM sodium phosphate, dibasic; about 0.05% w/v polysorbate 80; about 25 mM L-arginine; and about 50 mg/mL sucrose;
(ii) normal saline (0.9% sodium chloride); and
(iii) instructions for use.

43. The therapeutic kit of claim 42, wherein the formulation is lyophilized.

44. A method for producing a stable aqueous solution comprising ravulizumab at a concentration of no more than about 10 mg/mL; about 33.1 mM sodium phosphate, monobasic;
about 16.5 mM sodium phosphate, dibasic; about 0.05% w/v polysorbate 80; about 25 mM L-arginine; about 50 mg/mL sucrose; and water for injection, the method comprising:
i) providing a first aqueous solution comprising about 10 mg/mL ravulizumab,
ii) subjecting the first aqueous solution to diafiltration into a formulation about 33.1 mM sodium phosphate, monobasic; about 16.5 mM sodium phosphate, dibasic; about 0.05% w/v polysorbate 80; about 25 mM L-arginine; about 50 mg/mL sucrose; and water for injection at pH 7.4 to thereby produce a second aqueous solution; and
iii) concentrating the second aqueous solution to produce a stable aqueous solution comprising ravulizumab at a concentration of about 100 mg/mL; about 33.1 mM sodium phosphate, monobasic; about 16.5 mM sodium phosphate, dibasic; about 0.05% w/v polysorbate 80; about 25 mM L-arginine; about 50 mg/mL sucrose; and water for injection.
